(19) United States
(12) Patent Application Publication
(10) Pub. No.: US 2008/0299228 A1
(43) Pub. Date: Dec. 4, 2008

(54) TOPICAL COMPOSITIONS COMPRISING A MACROMOLECULE AND METHODS OF USING SAME

(76) Inventors: Alan Gerald Harris, New York, NY (US); Keith Arthur Johnson, Durham, NC (US); Tatjana Lukic, New York, NY (US)

Correspondence Address: FISH & RICHARDSON PC, P.O. BOX 1022, MINNEAPOLIS, MN, 55440-1022, US

(21) Appl. No.: 12/129,615

(22) Filed: May 29, 2008

Related U.S. Application Data
(60) Provisional application No. 60/940,509, filed on May 29, 2007.

(51) Int. Cl. A61K 38/29; A61P 17/00

(52) U.S. Cl. 424/709; 514/12

(57) ABSTRACT

Compositions, including those in which the active ingredient or agent is stable and capable of penetrating the sites of topical application are provided. The compositions can include a macromolecule, for example, a parathyroid hormone (PTH), a PTH-like hypercalcemic factor (CFF) or biologically active variant of a PTH or CFF in a vehicle comprising a polymer or cationic liposome. The compositions can also include other substances that further promote the stability of the active agent (e.g., buffers, antioxidants or carbohydrates). Also provided are methods for using the compositions for treatment of skin disorders (e.g., hyperproliferative skin disorders, including, but not limited to, for example, psoriasis, acne, rosacea or acne.)

### CROSS-REFERENCE TO RELATED APPLICATIONS

This application claims the benefit of the priority date of U.S. provisional application No. 60/940,509, filed on May 29, 2007. The content of the prior provisional application is hereby incorporated by reference herein in its entirety.

### FIELD OF THE INVENTION

The present invention is generally directed to compositions, which may be formulated as a gel for topical administration and that contain a macromolecule, such as a parathyroid hormone. The compositions can also include a polymer and optional additional ingredients, such as a buffer and non-volatile solvent. The compositions can be used for treating a variety of disorders, including skin conditions such as psoriasis. The invention is also directed to methods for packaging, making, and using the compositions.

### BACKGROUND

Skin disorders encompass a wide range of diseases and conditions and are a major cause of disability and disfigurement for a significant portion of the world population. Various skin disorders, for example, psoriasis, are characterized by an abnormal proliferation of skin cells. For many patients with skin disorders, the available remedies consist primarily of palliative care rather than curative treatments. Many of the most effective agents, for example, systemic agents, used to treat severe skin disorders carry an increased risk of morbidity. There is a continuing need for safe and effective treatments for skin disorders.

### SUMMARY OF THE INVENTION

The present invention is based, in part, on our discovery of various formulations in which the active ingredient or agent is stable (e.g., stable at room temperature) and capable of penetrating the skin or other sites of application to the body. Moreover, solid and liquid components of the formulations can be compartmentalized or packaged separately, which further prolongs the shelf life and allows shipment over long distances without refrigeration prior to their combination and use.

While the invention is not so limited, the present compositions (which we may also refer to as formulations) include gels suitable for application to the skin, including wounded skin, the cornea, the epithelial and or mucosal linings of the nose, mouth, and respiratory tract and to the anogenital area. More specifically, the invention features compositions (e.g., a gel, cream, ointment, or salve for topical application) that include (a) a therapeutically effective amount of a parathyroid hormone (PTH), a PTH-like hypercalcemic factor (CFF), or a biologically active variant of PTH or CFF, and (b) a vehicle comprising a polymer or a cationic liposome. The PTH can be a full-length PTH or a fragment thereof (e.g., the biologically active fragments described below or a mutant thereof). The compositions are pharmaceutically acceptable (e.g., non-toxic) and can further include a buffer. Suitable buffers include the solutions conventionally defined as buffers (i.e., solutions containing either a weak acid and its salt or a weak base and its salt, which is resistant to changes in pH). As the present compositions can have an acidic pH (e.g., a pH between about 3.0 and 5.0), the buffers can include acids such as acetic acid, citric acid, lactic acid, or a combination thereof. More generally, the buffer can include any acid having a pKa within about one pH unit of the target pH (i.e., of the desired pH of the composition (e.g., a gel) administered).

The present compositions also include one or more polymers, which may also be referred to as gelling agents where they contribute to the texture or viscosity of the compositions. The polymers may be naturally or non-naturally occurring, and may be cellulose-based. For example, the polymer can be, or can include, a hydroxyethylcellulose (HEC), a hydroxypropylcellulose (HPC), a methylcellulose, a carboxymethyl-cellulose, a hydroxypropyl methyl cellulose, a polyvinyl alcohol, a polyvinylpyrrolidone, an alginate, a chitosan, or a carbomer. While anionic polymers may be used, our results indicate that, at least for PTH(1-34), cationic or uncharged polymers may produce more appealing compositions.

In addition to the active agent (e.g., the PTH described above or other macromolecules described below) and a polymer, the present compositions can further include one or more of: an antioxidant (e.g., sodium thiosulfate or methionine); a chelating agent (e.g., EDTA); an anti-microbial agent; a volatile solvent; a non-volatile solvent; an emollient; a humectant; and a stabilizer. The anti-microbial agent can be phenoxyethanol, methylparaben, ethylparaben, or propylparaben; the volatile solvent can be water, ethanol, methylene chloride or isopropyl alcohol; the non-volatile solvent can be hexylene glycol, diethylene glycol monoethyl ether, glycerin, polyethylene glycol, dimethyl isosorbide, propylene carbonate, propylene glycol, or 1,2,6-trihydroxyhexane; the humectant can be hexylene glycol; and the stabilizer can be sorbitol, mannitol, raffinose, trehalose, or lactose. One of ordinary skill in the art would understand that a particular agent may reasonably satisfy the function of more than one of the types of ingredients included in the present compositions. For example, the same agent (e.g., hexylene glycol) may be included to serve as either a solvent or a humectant. Similarly, water may be included as a diluent and yet have some buffering capacity. In some embodiments, the compositions may be salt-free.

While several of our studies have used a PTH as the active agent, the invention is not so limited. The present compositions can include a therapeutically effective amount of any macromolecule (e.g., any molecule having a molecular weight of about 1,500 daltons to about 1,000,000 daltons) and a polymer. The macromolecule can be one that achieves a therapeutic or cosmetic benefit when applied to the skin or other area of the patient's body. Compositions that include such a macromolecule and a polymer (e.g., a cellulose-based polymer such as HEC) can further include one or more of: a solvent/humectant; a preservative; a buffer; a diluent; an antioxidant; a chelating agent; an anti-microbial agent; an emollient; and a stabilizer. In a particular embodiment, the present compositions include, and may include only, the active agent, a polymer, a solvent, a humectant, a preservative, a buffer and a diluent.

In a particular embodiment, the present compositions include a macromolecule that is present at about 0.005 to 0.5% (w/w).

The stable and penetrating compositions we have developed may serve to deliver a very wide variety of macromolecules. These include polypeptides, steroids, and other chemical and biological therapeutics. For example, the macromolecule can be a growth factor, interleukin, cytokine, antibody, enzyme, or hormone. While the macromolecule may be in a pure or substantially pure state prior to inclusion, the present compositions can also be used to deliver mixtures of macromolecules, such as those obtained from plant or cell extracts, homogenates, or lysates.

More specifically, the macromolecule can be an interferon such as interferon α-2b, which is useful in treating lesions on ano-genital tissues caused, for example, by a human papilloma virus (HPV) infection. The present compositions, when formulated for topical/mucosal administration are intended to facilitate transport of the active agent across the surface of the skin/mucosa, providing better access to the cells of the underlying basal epidermal layers (where viral infection predominates).

While various diseases and disorders can be treated with the present compositions, certain macromolecules known in the art (namely, those active in treating wrinkles and fine lines; firming skin tissue; and improving the radiance of the skin) can be included for cosmetic application (in which case the present compositions will include cosmetically effective amounts of the active agent). Alpha interferons can be used for cosmetic purposes as well as in the treatment of atopic eczema.

As noted, the macromolecule can be a growth factor. For example, a member of the transforming growth factor family (e.g., TGF-β3) can be included and used in the treatment or prevention of chemotherapy-induced oral mucositis in patients with lymphomas or solid tumors. TGF-β2 can be used to treat wounds and skin ulcers, such as those caused by diabetes. Epidermal growth factors can also be included for any type of wound healing. Other useful growth factors include the platelet-derived growth factors, nerve and glial growth factors, fibroblast growth factors and insulin-like growth factors (e.g., IGF-I and IGF-II).

In other instances, the macromolecule can be an immunosuppressant, such as cyclosporine or a macrolide immunosuppressant. The macrolides are a relatively new class of compounds sharing a macrolide-like structure and potent immunosuppressive activity in vitro and in vivo. Tacrolimus, the best known substance of this group, is registered in many countries for the prevention of transplant rejection, and this and other macrolide immunosuppressants, including other topical calcineurin inhibitors, are also effective in treating dermatological disorders (e.g., eczema). Due to their chemical structure, these compounds can be used for various topical/body surface treatments. The immunosuppressant cyclosporine improves tear production and can be used in the present formulations for dry eyes, allergic contact dermatitis, and conjunctival manifestations of actinic prurigo. Cyclosporin can also be used as formulated herein for the treatment of posterior blepharitis (styes).

In other instances, the macromolecule can be an interleukin. For example, the present formulations can include IL-18 and can be used in to treat skin damaged by uv-radiation. This includes sunburned skin, inflamed skin, and prematurely aging skin. IL-1 receptor antagonists can be included to inhibit inflammatory cell infiltration into the cornea.

Heparin can be included and used to help re-absorb haematomas, to treat burns and thrombophlebitis occurring close to the skin, and for relief of superficial inflammation. As heparin assists in strengthening and supporting the connective tissues, heparin can be used in cosmetic formulations and to treat scar tissue (promoting softness and elasticity). Heparin also has an antiphlogistic and anti-exudative effect, thus alleviating pain and promoting tissue metabolism and the process of healing.

Those of ordinary skill in the art will recognize many other specific macromolecules that can be included in the present formulations. Generally, these can be selected for treatment of circulatory disorders; superficial phlebitis and thrombophlebitis; varicose veins, indolent ulcers and bed sores; external haemorrhoids; wounds caused by accidents and injuries; sprains, strains, contusions and burns; haematomas; scar tissue (to prevent formation of keloid tissue); and bone and/or joint pain. For example, glucosamines and chondroitin can be used to relieve arthritic pain in the hands, feet, back, or joints.

Antibiotics can also be included as the active agent, as can a chemotherapeutic agent. For example, erythromycin can be formulated as described herein and used for the treatment of acne.

The kits of the present invention can include: (a) a solid component comprising a stabilized, solid PTH formulation (e.g., a formulation substantially free of any liquid), (b) a liquid component comprising an aqueous buffer and, optionally, an excipient to stabilize the formulation, and (c) instructions for combining the solid component and the liquid component. The solid component may further include a stabilizer in solid form (e.g., sorbitol, mannitol, raffinose, trehalose, or lactose) and/or a disintegrant in solid form (e.g., alginic acid, carboxy-methylcellulose salts, croscarmellose sodium, guar gum, microcrystalline cellulose, pregelatizined starch, sodium alginate, or prolacilin sodium). The liquid component can include an acid or a humectant and the solid component can include a salt.

While the present methods are not so limited, they include treating a subject who has a skin condition by administering to the subject, on an affected area (e.g., an area of the skin), any of the compositions described herein that include a suitable therapeutic agent. The subject can be a human, but veterinary uses are clearly also intended.

Treatable conditions particular to the skin include psoriasis, acne, comedones, polymorphonuclear leukocytes, rosacea, nodulocystic acne, acne conglobata, senile acne and secondary acne such as solar, medication-related, professional acne, ichthyosis, ichthyosiform states, Darier's disease, palmoplantar keratoderma, leukoplasias and leukoplasiform states, and cutaneous or mucous (buccal) lichen.

In any of the methods of treatment or in any of the “use” applications for the present compositions, one may carry out the step of identifying a subject in need of treatment (e.g., by diagnostic tests and other procedures).

As used herein, and unless otherwise specified, a “therapeutically effective amount” of a compound is an amount sufficient to provide a therapeutic benefit in the treatment or management of a disease or condition, or to delay or minimize one or more symptoms associated with the disease or condition. A therapeutically effective amount of a compound/agent means an amount of therapeutic agent, alone or in combination with other therapies, which provides a therapeutic benefit in the treatment or management of the disease or condition or a symptom thereof. The term “therapeutically effective amount” can encompass an amount that improves overall therapy, reduces or eliminates symptoms or causes of a disease or condition, or enhances the therapeutic efficacy of another therapeutic agent.

As used herein, and unless otherwise indicated, the term “PTH” refers to a mammalian or non-mammalian parathyroid hormone and its variants, analogs, derivatives and fragments, as well as PTH related proteins (PTHrP). The term “PTH” can encompass human PTH (hPTH) in some embodiments, and the variants can include mutants of PTH or mutants of a PTH fragment (e.g., a mutant in which 1-10 amino acid residues (e.g., 1, 2, or 3 residues) differ from those in the corresponding wild-type PTH by virtue of an addition, deletion, or substitution mutation). The term “xPTH(______-______)” refers to fragment of a PTH molecule of a particular mammal, with “x” signifying the mammal and “(______-______)” indicating the amino acid positions of the termini of the fragment.

As used herein, and unless otherwise indicated, the term “topical vehicle” refers to a vehicle comprising one or more constituents used for topical delivery of a PTH.

As used herein, and unless otherwise indicated, the term “cationic liposome” refers to a positively charged, synthetic, vesicle with an aqueous core enclosed in one or more surfactant layers.

As used herein, and unless otherwise indicated, the terms “individual”, “subject” or “patient” can be used interchangeably and are not limited to an individual under the care of a physician. In one embodiment, the subject or patient is a human.

The terms “treat,” “treating” and “treatment,” as used herein, contemplate an action that occurs while a patient is suffering from a specified disease or disorder, which reduces the severity of the disease or disorder, or retards or slows the progression of the disease or disorder.

### BRIEF DESCRIPTION OF THE DRAWINGS

FIG. 1 is a graph depicting assay values for PTH (1-34) formulations after storage at 40° C.

FIG. 2 is a graph depicting the effect of freezing method on the temperature profile for an HEC gel. The units on the x-axis are time in minutes; the units on the y-axis are degrees Celsius.

FIG. 3 is a graph depicting assay values for PTH (1-34) formulations after storage at 25° C.

FIG. 4 is a graph depicting the percutaneous absorption flux profiles of [¹²⁵I]-EGF, BSA, and Somatostatin. Each data point represents the Mean from three Donors expressed as ng/cm2/hr.

FIG. 5 is a graph depicting the total absorption and mass balance results across skin donors as percutaneous absorption of [¹²⁵I] BSA, Somatostatin and EGF through intact human cadaver skin over 48 hours from a Single Application. Mean±SE as percent of applied dose.

FIG. 6 illustrates the Franz chamber. A. depicts the Chamber Chimney (open to environment); B. depicts the Receptor Solution Compartment (4.5-6.5 mL); C. depicts the O-ring seal; D. depicts the skin (1.0 cm²); E. depicts the Heated Water Jacket; F. depicts the Magnetic Stir Bar; and G. depicts the Sampling Port.

### DETAILED DESCRIPTION

Compositions, including those in which the active ingredient or agent is stable and capable of penetrating the sites of topical application are provided. The compositions can include a macromolecule, for example, a parathyroid hormone (PTH), a PTH-like hypercalcemic factor (CFF) or biologically active variant of a PTH or CFF in a vehicle comprising a polymer or cationic liposome. The compositions can also include other substances that further promote the stability of the active agent (e.g., buffers, antioxidants or carbohydrates). Also provided are methods for using the compositions for treatment of skin disorders (e.g., hyperproliferative skin disorders, including, but not limited to, for example, psoriasis, acne, rosacea or acne.) The components of these compositions and methods for their use are described further below.

Macromolecules: Suitable macromolecules for use in the compositions described herein include, but are not limited to, fragments, derivatives, and variants of hormones, polyamino acid, polyamino acid derivative, peptides, proteins, glycoproteins, oligonucleotides, antibodies, glucosaminoglycans, steroids, antibacterial proteins, antiviral proteins, antifungal proteins, macrolides, enzymes, growth factors, growth factor receptors, interferons, interleukins, chemokines, cytokines, colony stimulating factors and chemotactic factors, transcription and elongation factors, cell cycle control proteins including, but not limited to, kinases and phosphatases, DNA repair proteins, apoptosis-inducing genes, apoptosis-inhibiting genes, oncogenes, antisense oncogenes, tumor suppressor genes, angiogenic and anti-angiogenic proteins, immune response stimulating and modulating proteins, cell surface receptors, and accessory signaling molecules and transport proteins. Examples of suitable macromolecules containing an amino acid include, without limitation, alpha-interferon, epidermal growth factor, transforming growth factor alpha, transforming growth factor beta, cyclosporin, somatostatin, growth hormone releasing peptides such as, but not limited to, growth hormone releasing peptide-6 (GHRP-6), or immunoglobulins. Examples of suitable glucosaminoglycans include, but are not limited to, heparin and glucosamine. Additional examples of suitable macromolecules include, without limitation, fluticasone propionate, clindamycin phosphate, neomycin, erythromycin, polymyxin B sulfate, ketoconazole, tacrolimus, and papain.

In certain embodiments, the molecular weight of the macromolecule is between about 500 daltons to about 1,000,000 daltons. In other embodiments, the molecular weight of the macromolecule is between about 1,500 daltons to about 1,000,000 daltons. In certain embodiments, the molecular weight of the macromolecule is between about 1,750 daltons to about 750,000 daltons, about 2,000 daltons to about 500,000 daltons, about 2,250 daltons to about 250,000 daltons, about 2,500 daltons to about 125,000 daltons, about 2,750 daltons to about 75,000 daltons, or about 3,000 daltons to about 50,000 daltons.

Parathyroid Hormones (PTHs): Suitable PTHs for use in the present compositions include fragments, analogs, derivatives, and variants of mammalian (e.g., human) parathyroid hormones as well as PTH related proteins (PTHrP). The PTH can be naturally occurring, or partially or completely synthetic, e.g. recombinant. In some embodiments, the PTH can be a peptide that is at least 3, and more preferably at least 8, amino acids long. In some embodiments, the PTH has at least about 10% sequence identity with a region of a mammalian PTH, such as a human PTH (hPTH). The PTH can be capable of inhibiting proliferation or enhancing the differentiation of keratinocytes. In some embodiments, the concentration of the PTH in the composition can range from about 0.05 mg/mL to about 50 mg/mL. Examples of suitable PTH include without limitation hPTH(1-84), hPTH(1-41), hPTH(1-38), hPTH(1-37), hPTH (1-34), hPTH (3-34), hPTH (5-34), hPTH(5-36), hPTH(7-34), hPTHrP(1-34), hPTHrP(1-141); hPTHrP(1-139), and hPTHrP(1-173). Also, calcemic factor fragments (CFF) such as CFF(1-34) and CFF(7-34) can be used. Other examples of suitable PTHs or CFFs are described in U.S. Pat. Nos. 5,527,772; 5,840,690; 6,066,618; U.S. patent application Ser. No. 10/311,366; U.S. patent application Ser. No. 11/639,238; Canadian Patent No. 1,326,460; Canadian Patent Application No. 2,413,860; European Patent No. 0415924; European Patent Application No. 01946366.5; German Patent Application No. 3855331; Japanese Patent Application No. 2802084; and International Publication Nos. WO 01/98,348 and WO 89/03,873.

Polymers: Suitable polymers useful in the present compositions are preferably ones that are biocompatible, i.e., chemically and physically compatible. Examples of suitable polymers include, but are not limited to, cellulose polymers or derivatives. Examples of cellulose polymers or derivatives include, but are not limited to, cellulose butyrate, cellulose acetate butyrate, cellophane, cellulose nitrate, cellulose propionate, cellulose ethers, hydroxypropylcellulose, hydroxypropylmethyl cellulose, hydroxypropylethyl cellulose, hydroxypropylbutyl cellulose, hydroxypropylpentyl cellulose, hydroxyethylcellulose, ethylcellulose, carboxymethylcellulose, hydroxypropylmethyl cellulose phthalate, methylcellulose, starch, and combinations thereof. Other suitable polymers include polyvinyl alcohol, polyvinylpyrrolidone, xantham gum, carrageenan, alginates, chitosan, or carbomer. In certain embodiments, the polymer can be a neutral polymer, i.e. the polymer does not have a charge. Examples of such polymers include, but are not limited to, the cellulose polymers discussed above.

Cellulose, cellulose floc, powdered cellulose, microcrystalline cellulose, silicified microcrystalline cellulose, carboxyethylcellulose, carboxymethylcellulose, hydroxyethylcellulose, methylhydroxyethylcellulose, hydroxymethylcellulose, hydroxypropylcellulose, hydroxypropylmethylcellulose, hydroxypropyl-methylcellulose phthalate, ethylcellulose, methylcellulose, carboxymethylcellulose sodium, and carboxymethyl cellulose calcium include, but are not limited to, those described in R. C. Rowe and P. J. Shesky, Handbook of pharmaceutical excipients, (2006), 5th ed. As used herein, cellulose refers to natural cellulose. The term “cellulose” also refers to celluloses that have been modified with regard to molecular weight and/or branching, particularly to lower molecular weight. The term “cellulose” further refers to celluloses that have been chemically modified to attach chemical functionality such as carboxy, hydroxyl, hydroxyalkylene, or carboxyalkylene groups. As used herein, the term “carboxyalkylene” refers to a group of formula -alkylene-C(O)OH, or salt thereof. As used herein, the term “hydroxyalkylene” refers to a group of formula -alkylene-OH.

Suitable powdered celluloses for use in the invention include, but are not limited to Arbocel (available from JRS Pharma), Sanacel (available from CFF GmbH), and Solka-Floc (available from International Fiber Corp.).

Suitable microcrystalline celluloses include, but are not limited to, the Avicel pH series (available from FMC Biopolymer), Celex (available from ISP), Celphere (available from Asahi Kasei), Ceolus KG (available from Asahi Kasei), and Vivapur (available from JRS Pharma).

As used herein, the term “silicified microcrystalline cellulose” refers to a synergistic intimate physical mixture of silicon dioxide and microcrystalline cellulose. Suitable silicified microcrystalline celluloses include, but are not limited to, ProSolv (available from JRS Pharma).

As used herein, the term “carboxymethylcellulose sodium” refers to a cellulose ether with pendant groups of formula Na+ —O—C(O)—CH2-, attached to the cellulose via an ether linkage. Suitable carboxymethylcellulose sodium polymers include, but are not limited to, Akucell (available from Akzo Nobel), Aquasorb (available from Hercules), Blanose (available from Hercules), Finnfix (available from Noviant), Nymel (available from Noviant), and Tylose CB (available from Clariant).

As used herein, the term “carboxymethylcellulose calcium” refers to a cellulose ether with a pendant groups of formula —CH2-O—C(O)—O-½ Ca2+, attached to the cellulose via an ether linkage.

As used herein, the term “carboxymethylcellulose” refers to a cellulose ether with pendant carboxymethyl groups of formula HO—C(O)—CH2-, attached to the cellulose via an ether linkage. Suitable carboxymethylcellulose calcium polymers include, but are not limited to, Nymel ZSC (available from Noviant).

As used herein, the term “carboxyethylcellulose” refers to a cellulose ether with pendant carboxymethyl groups of formula HO—C(O)—CH2-CH2-, attached to the cellulose via an ether linkage.

As used herein, the term “hydroxyethylcellulose” refers to a cellulose ether with pendant hydroxyethyl groups of formula HO—CH2-CH2-, attached to the cellulose via an ether linkage. Suitable hydroxyethylcelluloses include, but are not limited to, Cellosize HEC (available from DOW), Natrosol (available from Hercules), and Tylose PHA (available from Clariant).

As used herein, the term “methylhydroxyethylcellulose” refers to a cellulose ether with pendant methyloxyethyl groups of formula CH3-O—CH2-CH2-, attached to the cellulose via an ether linkage. Suitable methylhydroxyethylcelluloses include, but are not limited to, the Culminal MHEC series (available from Hercules), and the Tylose series (available from Shin Etsu).

As used herein, the term “hydroxypropylcellulose”, or “hypomellose”, refers a cellulose that has pendant hydroxypropoxy groups, and includes both high- and low-substituted hydroxypropylcellulose. In some embodiments, the hydroxypropylcellulose has about 5% to about 25% hydroxypropyl groups. Suitable hydroxypropylcelluloses include, but are not limited to, the Klucel series (available from Hercules), the Methocel series (available from Dow), the Nisso HPC series (available from Nisso), the Metolose series (available from Shin Etsu), and the LH series, including LHR-11, LH-21, LH-31, LH-20, LH-30, LH-22, and LH-32 (available from Shin Etsu).

As used herein, the term “methyl cellulose” refers to a cellulose that has pendant methoxy groups. Suitable methyl celluloses include, but are not limited to Culminal MC (available from Hercules).

As used herein, the term “ethyl cellulose” refers to a cellulose that has pendant ethoxy groups. Suitable ethyl celluloses include, but are not limited to Aqualon (available from Hercules).

Cationic Liposomes: The cationic liposomes that can be used in the present compositions include, without limitation cationic surfactants or lipids. Suitable cationic surfactants or lipids include, but are not limited to, benzalkonium chloride, stearalkonium chloride, cetyl pyridinium chloride, 2-dioleoyl-3-trimethyl ammonium propane, dimethyl dioctadecyl ammonium bromide.

Additionally, the cationic liposomes can include polyoxyethylene fatty acid esters, polyoxyethylene fatty acid esters, polyoxyethylene sorbitan esters, polyoxyethylene glyceryl monoesters, polyoxyethylene glyceryl diesters, cetyl alcohol, stearyl alcohol, glyceryl monostearate, glyceryl distearate, sucrose distearate, and propylene glycol stearate, as well as cholesterol or derivatives thereof.

In some embodiments, the liposomes used in the compositions comprising PTH are anionic liposomes that comprise an anionic surfactant. Suitable anionic surfactants include but are not limited to, alkyl and alkyl ether sulfates, sulfated monoglycerides, sulfonated olefins, alkyl aryl sulfonates, primary or secondary alkane sulfonates, alkyl sulfosuccinates, acyl taurates, acyl isethionates, alkyl glycerylether sulfonate, sulfonated methyl esters, sulfonated fatty acids, alkyl phosphates, ethoxylated alkyl phosphates, acyl glutamates, acyl sarcosinates, alkyl sulfoacetates, acylated peptides, alkyl ether carboxylates, acyl lactylates, anionic fluorosurfactants, sulfonated fatty acids of the form R1-CH(SO₄)—COOH and sulfonated methyl esters of the from R1-CH(SO₄)—CO—O—CH₃, where R1 is a saturated or unsaturated, branched or unbranched alkyl group from about 8 to about 24 carbon atoms (e.g., alpha sulphonated coconut fatty acid and lauryl methyl ester); phosphates such as monoalkyl, dialkyl, and trialkylphosphate salts formed by the reaction of phosphorous pentoxide with monohydric branched or unbranched alcohols having from about 8 to about 24 carbon atoms (e.g., sodium mono or dilaurylphosphate, ethoxylated monoalkyl phosphates, etc.); acyl glutamates corresponding to the formula R1CO—N(COOH)—CH₂CH₂—CO₂M wherein R1 is a saturated or unsaturated, branched or unbranched alkyl or alkenyl group of about 8 to about 24 carbon atoms, and M is a water-soluble cation (e.g., sodium lauroyl glutamate and sodium cocoyl glutamate); alkanoyl sarcosinates corresponding to the formula R1CON(CH₃)—CH₂CH₂—CO₂M wherein R1 is a saturated or unsaturated, branched or unbranched alkyl or alkenyl group of about 10 to about 20 carbon atoms, and M is a water-soluble cation (e.g., sodium lauroyl sarcosinate, sodium cocoyl sarcosinate, and ammonium lauroyl sarcosinate); alkyl ether carboxylates corresponding to the formula R1-(OCH₂CH₂)x-OCH₂—CO₂M wherein R1 is a saturated or unsaturated, branched or unbranched alkyl or alkenyl group of about 8 to about 24 carbon atoms, x is 1 to 10, and M is a water-soluble cation (e.g., sodium laureth carboxylate); acyl lactylates corresponding to the formula R1CO—[O—CH(CH₃)—CO]x-CO₂M wherein R1 is a saturated or unsaturated, branched or unbranched alkyl or alkenyl group of about 8 to about 24 carbon atoms, x is 3, and M is a water-soluble cation (e.g., sodium cocoyl lactylate); carboxylates, nonlimiting examples of which include sodium lauroyl carboxylate, sodium cocoyl carboxylate, and ammonium lauroyl carboxylate; anionic fluorosurfactants; and natural soaps derived from the saponification of vegetable and/or animal fats & oils examples of which include sodium laurate, sodium myristate, palmitate, stearate, tallowate, cocoate. Specific examples of surfactants that may be used are sodium dodecyl sulfate, ammonium, potassium, magnesium, or TEA salts of lauryl or myristyl sulfate.

Non-Volatile Solvents: Suitable non-volatile solvents for use in the compositions are ones that allow the PTH to be delivered to or penetrate the skin. The solubility of the PTH in the solvent is determined by combining a known weight of the PTH and a known weight or volume of the solvent. The solubility can be measured as weight of the PTH (e.g., mg) per weight of the solvent (e.g., grams). In some embodiments, the solubility of the PTH in the non-volatile solvent can be greater than about 1,000 mg/g. In some embodiments, the solubility of the PTH in the non-volatile solvent can be less than about 1,000 mg/g. For example, the solubility of the PTH in the non-volatile solvent can be less than about 500 mg/g, less than about 450 mg/g, less than about 400 mg/g, less than about 350 mg/g, less than about 300 mg/g, less than about 250 mg/g, less than about 200 mg/g, less than about 150 mg/g, less than about 100 mg/g, less than about 50 mg/g, less than about 40 mg/g, less than about 30 mg/g, less than about 20 mg/g, less than about 10 mg/g, less than about 5 mg/g, or less than about 1 mg/g. In some embodiments, the solubility of the PTH in the non-volatile solvent is between about 0.1 mg/g to about 5 mg/g. In other embodiments, the solubility of the PTH in the non-volatile solvent is between about 0.5 mg/g to about 1 mg/g.

Non-volatile solvents and emollients that can be used in the present compositions include, but are not limited to, vegetable oils; esters, such as octyl palmitate, C₁₂ to C₁₅ alkyl benzoates, isopropyl myristate and isopropyl palmitate; ethers, such as dicapryl ether and dimethyl isosorbide; fatty alcohols, such as cetyl alcohol, stearyl alcohol and behenyl alcohol; non-volatile silicones, such as dimethicone and polysiloxanes; hydrocarbon oils, such as mineral oil, petrolatum, isoeicosane and polyisobutene; polyols, such as polyethylene glycol, propylene glycol, diethylene glycol monoethyl ether, butylene glycol, pentylene glycol and hexylene glycol; and waxes, such as beeswax and botanical waxes; glycerin, propylene carbonate, 1,2,6-trihydroxyhexane and combinations thereof.

Additional Constituents: The compositions of the present invention can also include additional constituents. These additional constituents include, without limitation buffers, acids, antioxidants, chelating agents, anti-microbial agents, additional solvents, emollients, or a combinations thereof. Such additional constituents can function, without limitation as: solvents, humectants, buffers, surfactants, thickening agents, preservatives, and permeation enhancers for the compositions described herein.

Suitable buffers include, but are not limited to, organic acid salts such as acetate, citrate, lactate, benzoate, tartrate, ascorbate, gluconate, carbonate, succinate, or phthalate; trishydroxylaminomethane (“Tris”), tromethamine hydrochloride; phosphate buffers or combinations thereof.

Suitable acids include, but are not limited to, acetic acid, citric acid, lactic acid, benzoic acid, tartaric acid, hydrochloric acid, phosphoric acid, and nitric acid.

Suitable antioxidants include, but are not limited to, sodium bisulfite, sodium metabisulfite, sodium thiosulfate, acetyl cysteine, cysteine, thioglycerol, sodium sulfite, acetone sodium bisulfite, dithioerythreitol, dithiothreitol, thiourea, propyl gallate, methionine, erythorbic acid or combinations thereof.

Suitable chelating agents include, but are not limited to, ethylenediamine-tetraacetic acid (EDTA) and salts thereof such as disodium EDTA, tetrasodium EDTA and calcium disodium EDTA; diethylenetriaminepentaacetic acid (DTPA) and salts thereof; hydroxyethlethylenediaminetriacetic acid (HEDTA) and salts thereof; nitrilotriacetic acid (NTA), and salts thereof, and citric acid and salts thereof, such as sodium citrate.

Suitable anti-microbial agents include, but are not limited to, phenoxyethanol, methylparaben, ethylparaben, propylparaben, benzalkonium chloride, benzyl alcohol, chlorobutanol, metacresol, buylparaben, benzoic acid, sodium benzoate, imidurea, phenylethanol, cetylpyridinium chloride, methyl p-hydroxybenzoate, propyl p-hydroxybenzoate, timerosal or combination thereof.

Suitable additional solvents include, but are not limited to, ethanol, methylene chloride, isopropyl alcohol, castor oil, ethylene glycol monoethyl ether, diethylene glycol monobutyl ether, diethylene glycol monoethyl ether, dimethylsulfoxide, dimethyl formamide, tetrahydrofuran, and combinations thereof.

Suitable emollients include, but are not limited to, esters of fatty acids or alcohols and hydrocarbons, preferably C₈-C₂₀ alkyl ester of fatty acids such as, isopropyl myristate, isopropyl palmitate, isostearyl palmitate, tridecyl salicylate, C₁₂₋₁₅ octanoate and isopropyl stearate; mineral oil; dimethicone; cyclomethicone or combinations thereof.

Suitable humectants include, but are not limited to, glycerin, sorbitol, propylene glycol, hexylene glycol, lactic acid and salts thereof or combinations thereof.

Viscosity and pH of the Compositions: Preferably, the viscosity of the compositions are such that the compositions can be applied to the skin with no or minimal dripping. In some embodiments, the viscosity of the compositions is less than about 1 cps to greater than about 100,000 cps. In certain embodiments, the viscosity of the compositions is between about 100 cps to about 20,000 cps, about 250 cps to about 15,000 cps, about 500 cps to about 10,000 cps, about 750 cps to about 5,000 cps, or about 1,000 cps to about 3,000 cps. In certain embodiments wherein the compositions described herein are applied as a gel, the viscosity of the compositions can be between about 50 cps to about 500,000 cps, preferably about 100 cps to about 100,000 cps, or about 1,000 cps to about 20,000 cps.

The pH of the compositions described herein can be any value that will allow for stable compositions. In certain embodiments the pH of the compositions is acidic range. For example, in certain embodiments the pH of the compositions is between about 1 to about 6, or between about 2 to about 5, or between about 3 to about 5. In other embodiments, the pH of the compositions can be outside of this range, or slightly basic. In still other embodiments, the compositions described herein can have a neutral pH, i.e. having a pH of about 7.

An exemplary composition within the present invention is as follows:

| Component | Function | % w/w |
|---|---|---|
| PTH(1-34) | Active | 0.05 to 0.1 |
| Hexylene glycol, NF¹ | Solvent/Humectant | 12.00 |
| Hydroxyethylcellulose, NF | Gelling agent | 1.25 |
| Phenoxyethanol, BP | Preservative | 0.20 |
| Methylparaben, NF | Preservative | 0.15 |
| Ethylparaben, NF | Preservative | 0.05 |
| Acetic acid (glacial), USP | Buffer | 0.041 |
| Sodium acetate (anhydrous), USP | Buffer | 0.010 |
| Purified water, USP | Diluent | q.s. |

The amounts of one or more of these ingredients can be varied, up or down, by about 1%, 2%, 5%, 10%, 15% or 20%.

Administration and Packaging of the Compositions: The compositions of the present invention are designed to be administered topically. They can, however, also be administered via other routes such as, parenteral, subcutaneous, intramuscular, intraorbital, intracapsular, intraspinal, intrasternal, intravenous, intradermal, intraperitoneal, intraportal, intra-arterial, intrathecal, transmucosal, intra-articular, intrapleural, epidural, mucosal (e.g., intranasal), by injection or infusion, as well as by oral, inhalation, pulmonary, and rectal administration.

The compositions described herein can take the form of solutions, creams, water-in-oil emulsions, oil-in-water emulsions, ointments, or gels. For topical compositions in the form of solutions, the solutions can be administered by contacting the skin with such solution contained on a vehicle, such as but not limited to, a pledget (i.e., a pad, compress, or relatively flat piece of material for delivering a therapeutic agent). Topical compositions in the form of solutions can also be administered by spraying the solution onto the skin of the subject. In such embodiments, the solution can be contained in a spray container, such as but not limited to, an aerosol spray container or a non-aerosol pump container. When an aerosol spray container is used, a portion of the container corresponding to the headspace can be filled with an inert gas such as, but not limited to, nitrogen or argon.

Topical compositions described herein in the form of gels can also be administered by spraying. Alternatively, topical compositions in the form of gels or creams can be contained in tubes, dropper bottles, or other suitable containers and can be applied directly by applying the gel or cream to the skin by hand.

Methods of Making: The topical compositions described herein may be manufactured by combining the constituents and using means such as conventional mixing, high shear mixing, dissolving, granulating, dragee-making, levigating, emulsifying, encapsulating, entrapping, spray drying, or lyophilizing processes. For topical compositions in the form of solutions, the compositions may be manufactured by means of conventional mixing. Preferably, the mixing will take place at room temperature or below. For topical compositions in the form of gels or creams, the compositions may be manufactured by means of high shear mixing. Preferably, the high shear mixing will take place at an elevated temperature, wherein the PTH is added after the composition has cooled down.

Kits for Preparing Topical Compositions: The compositions described herein may be stabilized or further stabilized at room temperature through appropriate processing and packaging. For instance, in certain embodiments, the solid and liquid components of a kit used to prepare the topical composition are packaged in separate containers or closure systems. In such embodiments, both the solid and liquid components of the kit are stable at room temperature.

“Stable” means that the compositions are: within product specifications after six months of storage at 40° C. and/or after 12 months at 30° C. The present compositions are considered stable at room temperature where the results of product testing indicate a product within specification after six months at 25° C. and 60% relative humidity. A product that is tested after six months of storage at 40° C. and that is still found to be within specifications would generally indicate that the product is stable at room temperature for two years. Room temperature is 20° C. to 25° C.

At the time of dispensing, the solid and liquid components are combined by either adding the solid component to the liquid component, or adding the liquid component to the solid component, and then gently agitating or shaking the combination to produce a homogeneous and pharmaceutically-acceptable topical formulation. Such a combination may be stable at refrigerated or room temperature for at least 28 days after dispensing.

The solid component of the kit used to prepare the room temperature-stable topical compositions described herein includes a macromolecule, such as a PTH, in a stable solid form, which is substantially free of a liquid. In some embodiments, the solid component can include a PTH combined with one or more excipients including, but not limited to, polymers, cationic liposomes, antioxidants, chelating agents, or stabilizers or stabilizing agents in solid form such as, but not limited to, sorbitol, mannitol, raffinose, trehalose, and lactose. For example, to prepare a topical composition in the form of a gel, the solid component of the kit can include a polymer. In certain embodiments, the solid component may also include excipients to facilitate disintegration or dispersion of the liquid component such as, but not limited to, alginic acid, carboxymethylcellulose salts, croscarmellose sodium, guar gum, microcrystalline cellulose, pregelatinized starch, sodium alginate, or prolacrilin sodium.

The solid component as herein described may be prepared by conventional methods such as, but not limited to, lyophilization, lyophilization followed by milling, spray drying, or spray freezing, and may take various forms such as, but not limited to, a lyophilized cake, a powder, or a tablet. In any of these forms, the solid component may be packaged in a suitable container or closure system including, but not limited to, a sachet, a pouch, a pledget, a blister package, or a bottle.

The liquid component of the kit described herein may include an aqueous buffer plus other excipients required for stability and topical drug delivery including, but not limited to, solvents, antimicrobial agents, buffers, antioxidants, chelating agents, emollients, and humectants. The liquid component may be contained in tubes, dropper bottles, or other suitable containers or closure systems.

Methods of Treatment: The compositions and methods disclosed herein are useful for the treatment of skin disorders. Treatment can completely or partially abolish some or all of the signs and symptoms of the skin disorder, decrease the severity of the symptoms, delay their onset, or lessen the progression or severity of subsequently developed symptoms.

The formulations comprising PTH, CFF or a biologically active variant thereof are applicable to conditions characterized by an abnormal proliferation of skin cells, e.g., hyperproliferative skin disorders. These disorders may be the result of disease, an immune system disorder, infection, or of an unknown cause, and they may be influenced by one's genetic constitution. A patient who has a skin disorder associated with abnormal proliferation of skin cells is a candidate for treatment with the compositions described herein.

Skin disorders amenable to treatment may be characterized by abnormal keratinization. Normally, the growth of keratinocytes, or keratinization, occurs when skin cells move up through the various layers of skin, changing shape and composition as they differentiate and become filled with keratin, until the skin cells reach the surface and are sloughed off. Various skin disorders, such as psoriasis, are characterized by an increased proliferation rate of skin cells. The resulting several-fold increase in skin cell population reduces the turnover time of the epidermis from the normal 27 days to 3-4 days. Such a shortened interval prevents the normal cell maturation and keratinization processes from occurring. This failure of maturation is reflected in an array of abnormal morphologic and biochemical changes, such as scaling, skin lesions and, in some cases, internal damage such as arthritis.

Psoriasis most commonly appears as inflamed, edematous skin lesions covered with a silvery white scale. There are five types of psoriasis. Discoid/plaque psoriasis, the most common type, is characterized by raised inflamed lesions covered with a silvery white scale. The scale may be scraped away to reveal inflamed skin beneath. This is most common on the extensor surfaces of the knees, elbows, scalp, and trunk. The nails may be pitted and/or thickened. Guttate psoriasis presents as small red dots of psoriasis that usually appear on the trunk, arms, and legs; the lesions may have some scale. It frequently appears suddenly after an upper respiratory infection. Inverse psoriasis occurs on the flexural surfaces, armpit, groin, under the breast, and in the skin folds and is characterized by smooth, inflamed lesions without scaling. Pustular psoriasis presents as sterile pustules appearing on the hands and feet or, at times, diffusely, and may cycle through erythema, pustules, and scaling. Erythrodermic psoriasis presents as generalized erythema, pain, itching, and fine scaling.

Scalp lesions occur in approximately 50% of patients, presenting as erythematous raised plaques with silvery white scales on the scalp. Nail psoriasis may cause pits on the nails, which may develop yellowish color and become thickened. Nails may separate from the nail bed. Oral psoriasis may present with whitish lesions on the oral mucosa, which may appear to change in severity from day to day. It may also present as severe cheilosis with extension onto the surrounding skin, crossing the vermillion border. Psoriatic arthritis affects approximately 10% of those with skin symptoms. The arthritis is usually in the hands, feet, and, at times, in larger joints. It produces stiffness, pain, and progressive joint damage.

The compositions provided herein are also applicable to other skin disorders including, acne, acne vulgaris, nodulocystic acne, acne conglobata, senile acne and secondary acne such as solar, medication-related, professional acne, comedones, disorders associated with polymorphonuclear leukocytes, rosacea, ichthyosis, ichthyosiform states, Darier's disease, palmoplantar keratoderma, leukoplasias and leukoplasiform states, e.g., leukoplakias and erythroplakias, and cutaneous or mucous (buccal) lichen. Symptoms of acne include comedoes, e.g., whiteheads, blackheads, and pimples. These can occur on the face, neck, shoulders, back, or chest. In some instances, the naturally occurring largely commensual bacteria, Propionibacterium acnes, can cause inflammation, leading to inflammatory lesions (papules, infected pustules, or nodules) in the dermis around the microcomedo or comedo, which results in redness and may result in scarring or hyperpigrnentation. Acne lesions range in severity from comedones (blackheads and whiteheads) to nodules and cysts and include papules, a small (5 millimeters or less), solid lesions slightly elevated above the surface of the skin; pustule, dome-shaped, fragile lesions containing pus that typically consists of a mixture of white blood cells, dead skin cells, and bacteria; macules, temporary red spots left by a healed acne lesion; nodules, solid, dome-shaped or irregularly-shaped lesions characterized by inflammation that extends into deeper layers of the skin and may cause tissue destruction that results in scarring; and cysts, sac-like lesions containing liquid or semi-liquid material consisting of white blood cells, dead cells, and bacteria, that may be severely inflamed and extend into deeper layers of the skin, and can result in scarring. Cysts and nodules often occur together in a severe form of acne called nodulocystic.

Rosacea symptoms include erythema (flushing and redness) on the central face and across the cheeks, nose, or forehead but also less on the neck and chest. As rosacea progresses, other symptoms can develop such as semi-permanent erythema, telangiectasia (dilation of superficial blood vessels on the face), red domed papules (small bumps) and pustules, red gritty eyes, burning and stinging sensations, and in some advanced cases, a red lobulated nose (rhinophyma). Subtypes of rosacea include erythematotelangiectatic rosacea, characterized by flushing and persistent redness, and may also include visible blood vessels; papulopustular rosacea, characterized by persistent redness with transient bumps and pimples; phymatous rosacea, characterized by skin thickening, often resulting in an enlargement of the nose from excess tissue; and ocular rosacea, characterized by ocular manifestations such as dry eye, tearing and burning, swollen eyelids, recurrent styes and potential vision loss from corneal damage.

Ichthyosis and ichthyosiform states are a family of genetic skin diseases characterized by dry, thickened, scaling skin. The skin's natural shedding process is slowed or inhibited and, in some forms of ichthyosis, the production of skin cells is too rapid. There are estimated to be at least twenty-eight varieties of ichthyosis, with a wide range of severity and associated symptoms; the most common is Ichthyosis Vulgaris. Ichthyosis can be associated with one or more of the following: overheating, since many idividuals with ichthyosis do not sweat normally because the thickness of their skin does not allow their sweat to reach the surface of their skin and cool them effectively; limitations in movement due to tightness of the skin; secondary infection because splitting and cracking of the skin can lead to skin infections and/or systemic infections; and impaired eyesight or hearing due to skin build up over the eyes or ears. Examples of ichthyosis and ichthyosiform states include, without limitation, congenital ichthyosiform erythroderma, Darier's disease, epidermolytic hyperkeratosis, ichthyosis hystrix, ichthyosis bullosa of Siemens, b-CIE, bullous congenital ichthyosiform erythroderma, KID syndrome, keratitis, ichthyosis and deafness, lamellar ichthyosis, n-CIE; non-bullous congenital ichthyosiform erythroderma, Sjögren-Larsson Syndrome, X-linked Ichthyosis, steroid sulfatase deficiency, and recessive X-linked ichthyosis.

Leukoplakia is a white or gray patch that develops on the tongue or the inside of the cheek. It is the mouth's reaction to chronic irritation of the mucous membranes of the mouth. Leukoplakia patches can also develop on the female genital area. “Hairy” leukoplakia of the mouth is a form of leukoplakia that is seen only in people who are infected with HIV, have AIDS, or AIDS-related complex. It consists of fuzzy, hence the name “hairy,” white patches on the tongue and less frequently elsewhere in the mouth.

Palmoplantar keratoderma (PPK) constitutes a heterogeneous group of disorders characterized by thickening of the palms of the hands and the soles of the feet. The PPKs can initially be divided based on whether they are inherited or acquired. The inherited PPKs, include three distinct clinical patterns of epidermal involvement: diffuse PPK, which is uniform involvement of the palmoplantar surface; focal PPK, which consists of localized areas of hyperkeratosis located mainly on pressure points and sites of recurrent friction; and punctate keratoderma, which features multiple, small, hyperkeratotic papules, spicules, or nodules on the palms and soles. These tiny keratoses may involve the entire palmoplantar surface or may be restricted to certain locations (i.e., palmar creases). The keratodermas can then be further subdivided based on whether only an isolated keratoderma is present or whether other skin findings are present and/or other organs are involved. The first subclassification is simple keratoderma, which is isolated PPK. The second is keratodermas with associated features such as lesions of nonvolar skin, hair, teeth, nails, sweat glands, and/or with abnormalities of other organs. Acquired forms of PPK include for example, without limitation keratoderma climacterium, reactive/inflammatory PPK, infectious PPK, drug-related PPK, PPK with manifestations of systemic disease, and PPK associated with internal malignancy.

Cutaneous lichen or Lichen planus (LP) is a pruritic, papular eruption characterized by its violaceous color; polygonal shape; and, sometimes, fine scale. It is most commonly found on the flexor surfaces of the upper extremities, on the genitalia, and on the mucous membranes. LP is most likely an immunologically mediated reaction. Buccal or Oral lichen planus (OLP) is a chronic inflammatory disease that causes bilateral white striations, papules, or plaques on the buccal mucosa, tongue, and gingivae. Erythema, erosions, and blisters may or may not be present.

The formulations comprising EGF or a biologically active variant thereof are useful for wound healing and wound repair, for example, to stimulate regeneration of dermal and epidermal tissue. Wounds in need of repair can arise from diverse medical conditions, including, for example, congenital malformations, traumatic injuries, infections, and surgical resections and can include acute as well as chronic wounds, e.g., diabetic foot ulcers, decubitus ulcers, and venous stasis ulcers. The formulations comprising EGF or a biologically active variant thereof are also useful for the treatment and management of oral mucositis, particularly that induced by chemotherapy in cancer patients.

The formulations comprising somatostatin, somatostatin analogues or a biologically active variant thereof are useful for benign or malignant proliferative skin disease. Benign skin diseases include, for example, rosacea and reoccurring keratosis, e.g., pre-epitheliomatosis, actinic keratosis due to overexposure to the sun, and keratosis due to ageing. The formulations comprising somatostatin, somatostatin analogues or a biologically active variant thereof are also useful for topical relief of pain, particularly chronic pain such as experienced as burning or hyperesthesia. Malignant skin diseases include melanoma, malignant skin metastases of melanoma, reoccurring keratosis, non-invasive basal cellular epithelioma, pagetoid melanoma, and basal cell carcinoma. The somatostatin analogs may be administered directly to the diseased skin or may be used as a follow-up treatment after surgical excision or radiotherapy of the primary tumor to prevent reoccurrence. The formulations comprising somatostatin, somatostatin analogues or a biologically active variant thereof are useful for treatment of eye diseases, including thyroid eye disease (also known as TED or Graves' opthamolopathy) and cystoid diabetic macular edema.

The formulations comprising BSA or a biologically active variant thereof are useful for the prevention and therapy of periodontitis and diseases of the teeth and of the tissues of the oral cavity, topical antibiotic delivery, preparation (all with other compounds) for topical treatment of skin or eye diseases caused by viral infections, the preparation of interactive dressings.

Even more generally, the present compositions can be used to treat any condition that is responsive to the active agent. For example, an infection may be treated with compositions including an antibiotic; a site of tumor excision may be treated with a chemotherapeutic agent; and so forth.

Any composition described herein can be administered topically to any part of the subject's skin. In some embodiments, a composition can be delivered to, without limitation, the brain, the cerebrospinal fluid, joints, nasal mucosa, blood, lungs, intestines, muscle tissues, skin, or the peritoneal cavity of a mammal. In terms of routes of delivery, a composition can be administered by intravenous, intracranial, intraperitoneal, intramuscular, subcutaneous, intramuscular, intrarectal, intravaginal, intrathecal, intratracheal, intradermal, or transdermal injection, by oral or nasal administration, or by gradual perfusion over time. In a further example, an aerosol preparation of a composition can be given to a host by inhalation.

The dosage required will depend on the route of administration, the nature of the formulation, the nature of the patient's illness, the patient's size, weight, surface area, age, and sex, other drugs being administered, and the judgment of the attending clinician. Suitable dosages are in the range of 0.01-1,000 μg/kg Wide variations in the needed dosage are to be expected in view of the variety of the PTH, CFF and biologically active variants available and the differing efficiencies of specific formulations and various routes of administration. Variations in these dosage levels can be adjusted using standard empirical routines for optimization, as is well understood in the art. Administrations can be single or multiple (e.g., 2- or 3-, 4-, 6-, 8-, 10-, 20-, 50-, 100-, 150-, or more fold).

The duration of treatment with any composition provided herein can be any length of time from as short as one day to as long as the life span of the host (e.g., many years). For example, PTH, CFF and biologically active variants thereof can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present peptides can be administered once (or twice, three times, etc.) daily, weekly, monthly, or yearly.

An effective amount of any composition provided herein can be administered to an individual in need of treatment. The term “effective” as used herein refers to any amount that induces a desired response while not inducing significant toxicity in the patient. Such an amount can be determined by assessing a patient's response after administration of a known amount of a particular composition. In addition, the level of toxicity, if any, can be determined by assessing a patient's clinical symptoms before and after administering a known amount of a particular composition. It is noted that the effective amount of a particular composition administered to a patient can be adjusted according to a desired outcome as well as the patient's response and level of toxicity. Significant toxicity can vary for each particular patient and depends on multiple factors including, without limitation, the patient's disease state, age, and tolerance to side effects.

Any method known to those in the art can be used to determine if a particular response is induced. Clinical methods that can assess the degree of a particular disease state can be used to determine if a response is induced. For example, in a psoriasis patient, a reduction in psoriatic plaques, built-up scale, skin turn over, and a reduction in inflammation can be indicative of a dermatological response in a patient treated with the present peptides. For some disorders, blood or laboratory tests can be used to assist the clinician in evaluating a patient's response to the compositions. The particular methods used to evaluate a response will depend upon the nature of the patient's disorder, the patient's age, and sex, other drugs being administered, and the judgment of the attending clinician.

The compositions disclosed herein can include a single PTH, CFF or a biologically active variant thereof or can include a mixture of polypeptides that conform to the wild-type PTH amino acid sequence with a PTH that is a biologically active variant of the wild type sequence. For example, formulations of a PTH or CFF can be prepared as a combination of peptides of 2, 3, 4, 5, 6, 7, 8, 9, 10, 15, 10, 30, or more different amino acid sequences. Thus, for example, the compositions can include mixtures of polypeptides conforming to hPTH(1-84), hPTH(1-41), hPTH(l-38), hPTH(1-37), hPTH(1-34), hPTH(3-34), hPTH(5 34), hPTH(5-36), or hPTH(7-34). The formulation can also include a mixture of PTH, CFF or a biologically active variants thereof, one or more of which have been post-synthetically modified, for example a PTH(1-34) that has been amidated.

The formulations provided herein can be administered in conjunction with other therapeutic modalities to an individual in need of therapy. For example, the fragments of a PTH, CFF or biologically active variant thereof can be given prior to, simultaneously with or after treatment with other agents or regimes (e.g., a phototherapy regime).

The formulations can be administered in conjunction with other therapies for treating skin disorders such as standard, small molecule-type pharmaceutical agents, biopharmaceuticals (e.g., antibodies or antibody-related immunotherapies, siRNAs, shRNAs, antisense oligonucleotides and other RNA inhibitory molecules, microRNAs, and peptide therapeutics), surgery, phototherapy or in conjuction with any medical devices that may be used to assist the patient. Standard therapies for treating psoriasis can include, for example, topical treatments, light therapy and oral medications. Topical psoriasis treatments include, for example, moisturizers, low-potency and high potency topical corticosteroids, vitamin D analogues, e.g., Calcipotriene (Dovonex), coal-tar derivatives, e.g., Anthralin. (Dritho-Scalp or Psoriatec), topical retinoids, e.g., tazarotene (Tazorac), calcineurin inhibitors, and coal tar. Light therapy involves exposing the skin to controlled amounts of natural, i.e., sunlight and/or artificial light, e.g., artificial ultraviolet A (UVA) or ultraviolet B (UVB) light, also known as broadband UVB, narrowband UVB therapy, photochemotherapy, or psoralen plus ultraviolet A (PUVA). Photochemotherapy involves taking a light-sensitizing medication (psoralen) before exposure to UVA light. UVA light penetrates deeper into the skin than does UVB light, and psoralen makes the skin more sensitive to the effects of UVA exposure. Other light therapies include excimer laser, in which a controlled beam of UVB light is aimed at the psoriasis plaques to control scaling and inflammation and combination light therapy. Phototherapy may be combined with other therapeutic agents, e.g., retinoids coal tar (the Goeckerman treatment) or a coal tar bath and an anthralin-salicylic acid paste (the Ingram regimen). Oral medications for psoriasis include agents that reduce the production of skin cells, for example, retinoids, e.g., acitretin (Soriatane), methotrexate, methotrexate in combination with folic acid; anti-inflammatory agents, for example, azathioprine; immunosuppresants, for example, cyclosporine; hydroxyurea; immunomodulating agents, for example alefacept (Amevive), efalizumab (Raptiva), etanercept (Enbrel) and infliximab (Remicade).

The formulations can be administered in conjunction with other therapies for treating acne, for example topical bacteriocidal agents, e.g., benzoyl peroxide, triclosan, or chlor-hexidine gluconate; topical antibiotics, including, for example, erythromycin, clindamycin, stiemycin, or tetracycline; topical retinoids, including for example, tretinoin (Retin-A), adapalene (Differin), and tazarotene (Tazorac); oral antibiotics, including for example, erythromycin, the tetracycline antibiotics, e.g., tetracycline, oxytetracycline, doxycycline, minocycline, or lymecycline), and trimethoprim; oral retinoids, including, for example, isotretinoin (Accutane, Amnesteem, Sotret, Claravis); phototherapy and laser treatments.

The formulations can be administered in conjunction with other therapies for treating rosacea, including, for example, but not limited to, topical antibiotics, e.g., metronidazole (Metrocream, Metrogel, Noritate) and azelaic acid (Azelex, Finacea); oral antibiotics, e.g., tetracycline, minocycline, doxycycline and erythromycin; oral retinoids, e.g., isotretinoin (Accutane); and surgical methods.

### EXAMPLES

### Example 1

### Physical and Chemical Stability

Physical stability (based on appearance and viscosity) and chemical stability (based on pH) were determined for the various vehicle formulations listed in Table 1.

TABLE 1
Formulations for physical and chemical stability assessment.

| | | Volatile | Non-volatile | Non-volatile | | Diluent |
|---|---|---|---|---|---|---|
| | Polymer | solvent | solvent | solvent | Surfactant | Buffer, |
| Formulation | HEC, % | Ethanol, % | Glycerin, % | PG, % | BAC, % | qs |
| 1 | 0 | 0 | 10 | 0 | 0 | citrate¹ |
| 2 | 1.25 | 0 | 10 | 20 | 0 | citrate¹ |
| 3 | 0 | 20 | 10 | 20 | 0 | citrate¹ |
| 4 | 1.25 | 20 | 10 | 0 | 0 | citrate¹ |
| 5 | 0 | 0 | 20 | 20 | 0 | citrate¹ |
| 6 | 1.25 | 0 | 20 | 0 | 0 | citrate¹ |
| 7 | 0 | 20 | 20 | 0 | 0 | citrate¹ |
| 8 | 1.25 | 20 | 20 | 20 | 0 | citrate¹ |
| 9 | 0 | 20 | 10 | 20 | 0 | benzoate |
| 10 | 0 | 0 | 10 | 10 | 0 | acetate¹ |
| 11 | 0 | 0 | 10 | 10 | 0.2 | benzoate |
| 12 | 0 | 10 | 0 | 10 | 0.2 | acetate |

HEC: Hydroxyethylcellulose
PG: Propylene glycol
BAC: Benzalkonium chloride
¹0.1% phenoxyethanol added as a preservative

In Table 1, Formulations 1-8 represent a fractional factorial design for: polymer (hydroxyethylcellulose or HEC), volatile solvent (ethanol), non-volatile solvent (glycerin), and another non-volatile solvent (propylene glycol or PG). Both glycerin and propylene glycol were evaluated alone and in combination, since PG is less polar than glycerin. Formulations 1-8 all used a pH 4 citrate buffer. Additional buffers were evaluated in Formulations 9-12 along with a surfactant (benzalkonium chloride or BAC). All percentages are weight percentages of the formulation. After preparation and initial testing, the formulations in Table 1 were stored at the following conditions:

3 freeze/thaw cycles (3 days at −20° C., 4 days at 40° C.)

4 weeks at 40° C.

4 weeks at 25° C.

4 weeks refrigerated (2-8° C.)

After completing the storage times, all samples were assessed for appearance and pH. Formulations 2, 4, 6, and 8 which contain a polymer or are gels, were also assessed for viscosity.

All samples at all conditions showed no significant change in appearance. All formulations remained clear, colorless, and essentially free of particulate material. The results for pH and viscosity are shown in Tables 2 and 3, respectively.

TABLE 2
pH of formulations initially and after storage.

| Vehicle | | | 1-month | 1-month | 1-month |
|---|---|---|---|---|---|
| Formulation | Initial | Freeze/Thaw | 2-8° C. | 25° C. | 40° C. |
| 1 | 4.01 | 3.99 | 3.92 | 3.98 | 4.11 |
| 2 | 3.83 | 3.85 | 4.07 | 4.01 | 4.07 |
| 3 | 3.95 | 4.10 | 4.10 | 3.90 | 3.89 |
| 4 | 4.05 | 3.90 | 4.09 | 3.89 | 3.88 |
| 5 | 4.07 | 4.00 | 3.91 | 4.11 | 4.01 |
| 6 | 3.92 | 4.01 | 3.90 | 4.07 | 4.08 |
| 7 | 3.94 | 3.99 | 4.00 | 4.10 | 4.12 |
| 8 | 3.96 | 4.10 | 4.01 | 3.99 | 3.95 |
| 9 | 4.11 | 3.89 | 4.02 | 4.05 | 3.93 |
| 10 | 4.05 | 4.07 | 4.08 | 3.91 | 4.05 |
| 11 | 4.07 | 3.90 | 4.01 | 3.99 | 3.97 |
| 12 | 3.92 | 3.90 | 3.95 | 4.02 | 3.89 |

TABLE 3
Viscosity (centipoise) of prototype gel vehicles initially and
after storage.

| Vehicle | | | 1-month | 1-month | 1-month |
|---|---|---|---|---|---|
| Formulation | Initial | Freeze/Thaw | 2-8° C. | 25° C. | 40° C. |
| 2 | 2800 | 3100 | 3200 | 2900 | 2700 |
| 4 | 2500 | 2900 | 2600 | 2600 | 2700 |
| 6 | 2500 | 3000 | 2800 | 2700 | 3000 |
| 8 | 3300 | 3400 | 3600 | 3500 | 3100 |

There was no significant change in pH for any vehicle after 3 freeze/thaw cycles or 4 weeks storage at 2-8, 25, or 40° C. There was also no significant change in viscosity for the gels under these storage conditions. There was a slight trend for the viscosity to increase on storage, which can happen as the gel recovers from the shear during preparation.

### Example 2

### Evaluation of hPTH(1-34) Topical Compositions

The hPTH(1-34) formulations were made to assess their stability at refrigerated and accelerated conditions. The results of the stability study are shown in Table 4.

TABLE 4
Constituents in the hPTH(1-34) formulations.

| | 1 | | 2 | | 3 | |
|---|---|---|---|---|---|---|
| Component | (Solution) | | (Gel) | | (Solution) | |
| PTH(1-34) | 0.5 | mg/g | 0.5 | mg/g | 0.5 | mg/g |
| Benzalkonium | 0.2% | w/w | 0.2% | w/w | 0.2% | w/w |
| chloride¹ | | | | | | |
| Propylene glycol | — | | — | | 12.0% | w/w |
| Hexylene glycol | 12.0% | w/w | 12.0% | w/w | — | |
| Purified water | 21.6% | w/w | 20.35% | w/w | 6.6% | w/w |
| Hydroxyethylcellulose | — | | 1.25% | w/w | — | |
| Glycerin | — | | — | | 15.0% | w/w |
| Citrate buffer pH 4² | 66.0% | w/w | 66% | w/w | 66% | w/w |

¹The stock solution was 50% w/w surfactant, so 0.4% w/w was added to give 0.2% w/w.
²Contained 0.1% w/w phenoxyethanol

After preparing the formulations in Table 4, they were placed into glass vials and stored at 2-8 and 25° C. At specific time points, the assay for the amount of hPTH(1-34), in mg/g; the pH; and the appearance of the formulations were assessed. The results are summarized in Table 5.

TABLE 5
Summary of stability data for hPTH(1-34) formulations from Table 4.

| | Formulation | | |
|---|---|---|---|
| Condition | 1 | 2 | 3 |
| Initial | | | |
| Assay (% Initial) | 100.0 | 100.0 | 100.0 |
| pH¹ | 3.9-4.2 | 3.9-4.2 | 3.9-4.2 |
| Appearance | Conforms² | Conforms² | Conforms² |
| 1 week: 25° C. | | | |
| Assay (% Initial) | 99.7 | 99.5 | 99.4 |
| pH¹ | 3.9-4.2 | 3.9-4.2 | 3.9-4.2 |
| Appearance | Conforms² | Conforms³ | Conforms² |
| 2 weeks: 25° C. | | | |
| Assay (% Initial) | 99.2 | 99.6 | 98.9 |
| pH¹ | 3.9-4.2 | 3.9-4.2 | 3.9-4.2 |
| Appearance | Conforms² | Conforms³ | Conforms² |
| 1 Month: 25° C. | | | |
| Assay (% Initial) | 98.2 | 98.5 | 97.4 |
| pH¹ | 3.9-4.2 | 3.9-4.2 | 3.9-4.2 |
| Appearance | Conforms² | Conforms³ | Conforms² |
| 1 Month: 2-8° C. | | | |
| Assay (% Initial) | 99.7 | 100.0 | 99.8 |
| pH¹ | 3.9-4.2 | 3.9-4.2 | 3.9-4.2 |
| Appearance | Conforms² | Conforms³ | Conforms² |
| 2 Months: 2-8° C. | | | |
| Assay (% Initial) | 99.7 | 99.8 | 99.7 |
| pH¹ | 3.9-4.2 | 3.9-4.2 | 3.9-4.2 |
| Appearance | Conforms² | Conforms³ | Conforms² |

¹pH measured with color stick
²Clear, colorless solution essentially free of particulate matter
³Clear, colorless gel essentially free of particulate matter

The appearance for all formulations after one month of accelerated storage and after two months of refrigerated storage conformed to specification. There was no significant change in the pH, measured by color stick.

### Example 3

### Dermal Delivery Studies

Two groups of hPTH(1-34) formulations described below in Tables 6 and 7 were prepared for two screening studies (Screening Study 1 and Screening Study 2, respectively). In each study, the formulations described were applied topically to SKH-1 hairless mice for five days a week for two weeks in order to evaluate their pharmacological response. A vehicle control group was used for comparison purposes. The dosing volume for the hPTH(1-34) formulations was 0.05 mL, and the concentration was 0.05 mg/mL. At the end of each study, the mice were sacrificed and their skin was harvested, imbedded in paraffin, and sectioned. The skin sections were evaluated for epidermal thickness and the keratinocyte proliferation was compared to the control. The results are shown in Tables 6 and 7. All percentages are weight percentages of the formulation.

TABLE 6
Results for Screening Study 1

| | | Change in Keratinocyte |
|---|---|---|
| | | Proliferation Compared |
| Formulation | Composition | to Control |
| Control | Cream containing 0.0 mg/mL hPTH(1-34). | 0 |
| 1 | 0.5 mg/mL hPTH(1-34) solution containing 25% | −7.4% |
| | Transcutol¹ in pH 4 acetate buffer. | |
| 2 | 0.5 mg/mL hPTH(1-34) liposome formulation | −18% |
| | containing Generally Recognized as Safe (GRAS) | |
| | surfactants and manufactured using conventional | |
| | formulation technology. This formulation also | |
| | contained 12% hexylene glycol in a pH for acetate | |
| | buffer. | |
| 3 | 0.5 mg/mL hPTH(1-34) gel, containing 1.25% | −24% |
| | hydroxyethylcellulose, 12% hexylene glycol, 0.1% | |
| | phenoxyethanol in pH 4 acetate buffer. | |
| 4 | 0.5 mg/mL hPTH(1-34) solution containing 12% | −13% |
| | hexylene glycol in pH 4 acetate buffer. | |

¹Transcutol is diethylene glycol monoethyl ether.

TABLE 7
Results for Screening Study 2

| | | Change in Keratinocyte |
|---|---|---|
| | | Proliferation Compared |
| Formulation | Composition | to Control |
| Control | Cream containing 0.0 mg/mL hPTH(1-34). | 0 |
| 1 | 0.5 mg/mL hPTH(1-34) solution containing 12% | −31% |
| | hexylene glycol, 15% ethanol in pH 4 acetate | |
| | buffer. | |
| 2 | 0.5 mg/mL hPTH(1-34) solution containing Ion | −1% |
| | pair with PTH(1-34):SDS of 1:8 with 25% ethanol | |
| | and 25% propylene glycol in pH 4 acetate buffer. | |
| 3 | 0.5 mg/mL hPTH(1-34) solution containing Ion | +5% |
| | pair with PTH(1-34):SDS of 1:145 with 3% | |
| | hexylene glycol in pH 4 acetate buffer. | |
| 4 | 0.5 mg/mL hPTH(1-34) solution containing 12% | −16% |
| | hexylene glycol and 0.2% benzalkonium chloride | |
| | in pH 4 acetate buffer. | |
| 5 | 0.5 mg/mL hPTH(1-34) solution containing 12% | −32% |
| | propylene glycol in pH 4 acetate buffer. | |

### Example 4

### Dose Response Study

Formulation 3 as described above in Table 6 was used for a dose-response study to evaluate the effect of varying the level of hPTH(1-34). Four different formulations were prepared with levels of hPTH(1-34) varying from 0.05 to 1.0 mg/mL. The results are summarized in Table 8 below.

TABLE 8
Dose-response study results for gel formulation

| | Amount of | Change in Keratinocyte |
|---|---|---|
| Formulation | hPTH(1-34) | Proliferation Compared to Control |
| Control | 0.00 mg/mL | 0 |
| 1 | 0.05 mg/mL | 0 |
| 2 | 0.10 mg/mL | −31.9% |
| 3 | 0.50 mg/mL | −37.8% |
| 4 | 1.00 mg/mL | −49.6% |

### Example 5

### Dermal Permeation Study

Gel formulations containing hPTH(1-34) (based on Formulation 3 as described in Table 6 above) were prepared to assess permeation into the different layers of the skin. The quantitative formulations for the hPTH(1-34) gels used in the dermal permeation study are shown Table 9 below. All of the compositions are reported in % w/w.

TABLE 9
Quantitative formulations for hPTH(1-34) gels in a dermal
permeation study.

| | Formulation | | | |
|---|---|---|---|---|
| Component | 3-A | 3-B | 3-C | 3-D |
| hPTH(1-34) | 0.1 | 0.1 | 0.1 | 0.1 |
| Hexylene glycol | 12.00 | 12.00 | 12.00 | — |
| Propylene glycol | — | — | — | 12.00 |
| Hydroxyethylcellulose | 1.25 | 1.25 | 1.25 | 1.25 |
| Phenoxyethanol | 0.10 | 0.20 | 0.20 | 0.20 |
| Methylparaben | — | 0.15 | 0.15 | 0.15 |
| Ethylparaben | — | 0.05 | 0.05 | 0.05 |
| EDTA | — | — | 0.10 | — |
| Acetic acid (glacial) | 0.041 | 0.041 | 0.041 | 0.041 |
| Sodium acetate (anhydrous) | 0.010 | 0.010 | 0.010 | 0.010 |
| Purified water | q.s. | q.s. | q.s. | q.s. |

These formulations were applied to human cadaver skin mounted on diffusion (Franz) cells. The receptor phase contained aqueous phosphate buffer. Dosing was performed using a positive displacement pipette set to deliver 5 μL of formulation/cm² of skin.

The experiment was performed for 48 hours. At pre-selected times after dosing, the reservoir solution was removed in its entirety, replaced with fresh reservoir solution, and a pre-determined volume aliquot was saved for subsequent analysis. At the end of the experiment, the skin was washed to remove residual formulation, removed from the chamber, split into epidermis and dermis, and each was extracted. All samples were assayed by ELISA for hPTH(1-34) content. The results of the skin permeation study for the gel formulations are shown below in Table 10, where the numbers are reported as a percentage of the applied dose.

TABLE 10
hPTH(1-34) permeation into the skin for the gel
formulations listed in Table 9.

| Skin Layer | 3-A | 3-B | 3-C | 3-D |
|---|---|---|---|---|
| Dermis | 0.37% | 0.37% | 0.36% | 0.36% |
| Epidermis | 0.47% | 0.43% | 0.41% | 0.45% |
| Total in Skin | 0.84% | 0.80% | 0.78% | 0.81% |

Table 10 shows that the gel formulations delivered between 0.78 to 0.84% of the applied dose to the skin. The amount in the epidermis was higher than that in the dermis. There was no significant amount of hPTH(1-34) in the receptor phase. These results show that the gel formulations can consistently deliver significant amounts of HPTH(1-34) to the skin.

### Example 6

### Preparation of a Kit for Preparing Topical Compositions

There are various ways to prepare a kit that can be used to prepare topical compositions. Such ways include, but are not limited to, those described herein. For instance, a sachet can be prepared with 0.06 grams (g) of PTH(1-34) and 0.75 g of hydroxyethylcellulose as a homogenous powder that is stable at room temperature, prepared by spray drying. A bottle can be filled with the following: 7.2 g hexylene glycol, 0.12 g phenoxyethanol, 0.09 g methylparaben, 0.03 g ethylparaben, 0.06 g EDTA, 0.0246 g glacial acetic acid, 0.006 g anhydrous sodium acetate, and 51.6594 g of purified water. At the time of dispensing, the powder from the sachet is added to the bottle. The bottle is shaken to form a homogenous and pharmaceutically-acceptable gel for topical use.

Alternatively, 0.06 g of PTH(1-34) and 0.75 g of hydroxyethycellulose may be lyophilized in a bottle to form a cake that is stable at room temperature. Another bottle can be filled with the following: 7.2 g hexylene glycol, 0.12 g phenoxyethanol, 0.09 g methylparaben, 0.03 g ethylparaben, 0.06 g EDTA, 0.0246 g glacial acetic acid, 0.006 g anhydrous sodium acetate, and 51.6594 g of purified water. At the time of dispensing, the contents of the liquid bottle are added to the bottle holding the lyophilized cake. The bottle is shaken to form a homogeneous and pharmaceutically-acceptable gel for topical use.

Furthermore, a homogenous spray dried powder can be prepared with PTH(1-34), sorbitol, and hydroxyethylcellulose. The spray dried powder can be blended with pregelatinized starch then compressed to form a tablet. Each tablet contains 0.06 g of PTH(1-34), 0.75 g of hydroxyethylcellulose, 0.5 g sorbitol, and 0.15 g of pregelatinized starch. The tablet is then sealed in a blister package. A bottle can be filled with the following: 7.2 g hexylene glycol, 0.12 g phenoxyethanol, 0.09 g methylparaben, 0.03 g ethylparaben, 0.06 g EDTA, 0.0246 g glacial acetic acid, 0.006 g anyhydrous sodium acetate, and 51.0094 g of purified water. At the time of dispensing, the tablet is removed from the blister package and is added to the bottle. The bottle is shaken to form a homogeneous and pharmaceutically-acceptable gel for topical use.

Alternative preparations can include multiples of the amounts provided above or otherwise herein (i.e., the amounts can be doubled, tripled, and so forth).

### Example 7

### Formulations of Topical Compositions Comprising Macromolecules

A topical composition comprising EGF was prepared as indicated in Table 11. Topical compositions comprising glucosamine and phosphorothioate oligonucleotide are prepared according to the formulas in Tables 12, and 13, respectively.

TABLE 11
Quantitative formulation for topical composition
comprising macromolecules

| Component | % w/w |
|---|---|
| Epidermal growth factor | 0.1 |
| Hexylene glycol | 12.00 |
| Hydroxyethylcellulose | 1.25 |
| Sodium thiosulfate | 0.10 |
| Methylparaben | 0.15 |
| Propylparaben | 0.05 |
| Buffer | q.s. |

q.s. quantum sufficit

TABLE 12
Quantitative formulation for topical composition
comprising macromolecule.

| Component | % w/w |
|---|---|
| Glucosamine | 1.0 |
| Glycerin | 20.0 |
| Xantham gum | 0.75 |
| Sodium thiosulfate | 0.10 |
| Methylparaben | 0.15 |
| Propylparaben | 0.05 |
| Buffer | q.s. |

q.s. quantum sufficit

TABLE 13
Quantitative formulation for topical composition
comprising macromolecule.

| Component | % w/w |
|---|---|
| Phosphorothioate oligonucleotide | 0.01 |
| Sorbitol | 7.00 |
| Hydroxypropylcellulose | 0.75 |
| EDTA | 0.10 |
| Methylparaben | 0.15 |
| Propylparaben | 0.05 |
| Buffer | q.s. |

q.s. quantum sufficit

### Example 8

### Evaluation of Room Temperature Topical Gel Formulations of PTH(1-34): Materials and Methods

All excipients for the room temperature gel formulations met USP/NF monographs. Reagents for analysis were HPLC and/or ACS grade and nitrogen was ultra high purity (>99.999%). PTH(1-34) was supplied by Neo-MPS. Reverse phase HPLC RP-HPLC analysis of reconstituted gels/solutions was performed according to standard methods.

At each time point, two assays were performed on each formulation.

Viscosity was measured using a Brookfield LVF viscometer with a 13R sample chamber/34 spindle combination at 6 rpm.

Lyophilized gels were prepared by slowing adding the polymer to water with stirring. For gels with methylcellulose or hydroxypropylcellulose, the polymer was added to hot water and the product was cooled with stirring. The gels took between 1-3 hours to reach maximum viscosity. All gels were allowed to stand for at least 12 hours before any additional processing. If PTH(1-34) was included in the formulation, the peptide was added to an aliquot of gel and gently stirred for 10 minutes.

Prior to lyophilization, gels were filled into Type I glass vials and frozen. Freezing was performed on a freezer shelf (−20° C.) or in a dry ice/ethanol bath (˜−48° C.). The lyophilizer (Ultra-dry Labtop, Freezedry Specialties, Inc) was equipped with a condenser (−50° C.) and an ultra high vacuum pump (DUO 2.5, Pfeiffer Vacuum, Inc) to provide optimal conditions for sublimating the frozen water. No heating or cooling was applied to the lyophilizer shelf. Vials containing lyophilized gels with PTH(1-34) were purged with nitrogen before stoppering (Teflon coated butyl rubber) and sealing (aluminum crimp seal).

### Example 9

### Evaluation of Room Temperature Topical Gel Formulations of PTH(1-34): Effect of Various Polymers on Lyophilized Gel Rehydration Time

A panel of lyophilized polymer gel was screened for the capacity to reform gels following rehydration. Rehydration times were compared in lyophilized gels formed from five different neutral polymers: hydroxyethylcellulose (HEC), hydroxypropylcellulose (HPC), hydroxypropyl methylcellulose (HPMC), methylcellulose (MC) and polyvinyl alcohol (PVA). PTH(1-34) was not compatible with anionic polymers; combining PTH(1-34) with anionic polymers such as xanthan gum and carrageenan formed a cloudy/clotted gel.

Hydroxyethylcellulose (HEC), hydroxypropylcellulose (HPC), hydroxypropyl methylcellulose (HPMC), methylcellulose (MC) and polyvinyl alcohol (PVA) were used to prepare gels containing 2% w/w of the excipient according to the method in Example 8. The grade for each polymer was selected to match the thickening properties of HEC (Natrosol 250 M) as closely as possible. Five grams of gel were filled into vials that were lyophilized after freezing at −20° C. Within 18-20 hours, >99% of the water was removed. Gels were rehydrated by adding 5 grams of water. The results are summarized in Table 14.

TABLE 14
Results of initial screening for polymer rehydration time.

| Polymer | Grade | Rehydration time | |
|---|---|---|---|
| HEC | Natrosol 250M | 30-45 | minutes |
| HPC | Klucel MF | 60-90 | minutes |
| HPMC | Methocel F4M | >90 | minutes |
| MC | Methocel A4M | >90 | minutes |
| PVA | 40-45 mPas | >90 | minutes |

All five polymers formed stable lyophilized cakes in the vials. There was no evidence of collapse. Based on the results in Table 14, the polymers were ranked for rehydration as follows: HEC>HPC>HPMC, MC, PVA. HEC was selected for further analysis.

A panel of small carbohydrates was assayed for the ability to improve the stability of the lyophilized formulations. Mannitol, lactose, and sorbitol were added during the preparation of gels that were made with HEC according to the method of Example 8 except that the HEC was polymer grade 250HX; the final HEC 250× concentration in the wet gel was 1.125%. The lyophilized gels were evaluated qualitatively for the stability and the ease with which they could be rehydrated. The results of this study are summarized in Table 15. Stable cakes were obtained with gels that included mannitol or lactose; the inclusion of sorbitol resulted in unstable cakes that were difficult to rehydrate.

TABLE 15
Effect of adding small carbohydrates on lyophilized HEC gels.

| Gel Composition (wet) | Results of lyophilization |
|---|---|
| 1.125% HEC 250HX | Stable cake |
| No small carbohydrate | |
| 1.125% HEC 250HX | Cake partially collapsed, difficult to rehydrate |
| 1.0% Sorbitol | |
| 1.125% HEC 250HX | Cake collapsed, very difficult to rehydrate |
| 5.0% Sorbitol | |
| 1.125% HEC 250HX | Stable cake |
| 1.0% Mannitol | |
| 1.125% HEC 250HX | Stable cake |
| 1.0% Lactose | |

### Example 10

### Evaluation of Room Temperature Topical Gel Formulations of PTH(1-34): Stability of PTH(1-34) in Lyophilized Gels

Before preparing larger batches for longer term stability studies, three pilot batches were prepared on the 10 g scale to assess the solid product's stability. The batches included either PTH(1-34) plus HEC, PTH(1-34) plus HEC in 1.0% mannitol and PTH(1-34) plus HEC in 1.0% lactose. The lyophilized gels were prepared according to the method of Example 8. Stability was assayed according to by HPLC. The compositions and stability data are summarized in Table 16.

TABLE 16
Composition and stability data for lyophilized gels containing PTH(1-34).

| | | 2 week, | 4 week |
|---|---|---|---|
| Gel Batch Number/ | Initial assay, | 40° C. assay, | 40° C. assay, |
| Composition (wet) | % Initial | % Initial | % Initial |
| Batch 70712-1 | 100% | 99.2% | 98.8% |
| 1.125% HEC 250 HX | | | |
| 1.0% Mannitol | | | |
| 0.1% PTH(1-34) | | | |
| Batch 70712-2 | 100% | 95.2% | 90.4% |
| 1.125% HEC 250 HX | | | |
| 1.0% Lactose | | | |
| 0.1% PTH(1-34) | | | |
| Batch 70712-3 | 100% | 99.2% | 98.6% |
| 1.125% HEC 250 HX | | | |
| 0.1% PTH(1-34) | | | |

The PTH(1-34) formulations that included HEC alone (70712-3) or HEC in 1.0% mannitol (70712-1) showed less than 2% degradation after 1 month at 40° C., which was an acceptable level. The formulation that included lactose (70712-2) showed more than 10% degradation during the 4 week period, which is less than ideal.

The PTH(1-34) formulations that included HEC in 1.0% mannitol and HEC alone were prepared on a larger scale for use in a long-term stability analysis. In addition to these two lyophilized gel formulations, neat PTH(1-34), i.e., PTH powder, was also filled into vials (purged with nitrogen). The formulations for the longer stability study are summarized in Table 17.

TABLE 17
Composition of lyophilized formulations used in the 6 month stability
study for PTH(1-34).

| Batch Number | Wet Composition | Dry Composition | Comments |
|---|---|---|---|
| 70830-1 | 0.1% PTH(1-34) | ~8.2% PTH(1-34) | Stable cake |
| | 1.125% HEC 250HX | | structure |
| 70830-2 | 0.1% PTH(1-34) | ~4.5% PTH(1-34) | Stable cake |
| | 1.125% HEC 250HX | | structure |
| | 1.0% Mannitol | | |
| XF15// | Not applicable | Neat PTH(1-34) | Lyophilized |
| 339006-2 | | from Neo-MPS | powder |

PTH was assayed by HPLC analysis of reconstituted gels/solutions. The samples were assayed before storage, stored at 40° C. and assayed at 1, 3, and 6 months after storage. At each time point, 2 assays were performed on each formulation.

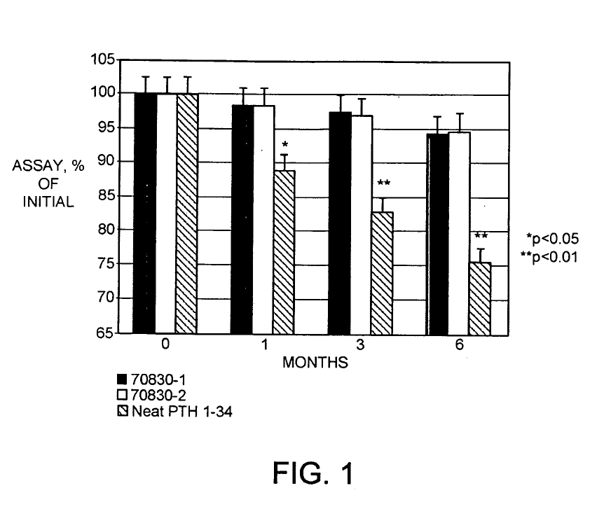

The results are shown in FIG. 1.

Both PTH(1-34) formulations in HEC alone (70830-1) and in 1.0% mannitol (70830-2) showed less than a 6% reduction over six months of storage. In contrast, neat PTH(1-34) (XF15//339006-2) showed significantly more degradation (about a 25% reduction) over 6 months even though its container/closure was purged with nitrogen. There was no significant difference in PTH(1-34) stability with or without mannitol. After six months of storage at 25° C., more than 98% of the starting material remained in both the 70830-1 and 70830-2 formulations. Neither 70830-1 nor 70830-2 contained NaCl. This was a component identified by Endo et al. (U.S. Pat. No. 5,563,122) as critical for PTH(1-34) stability in the solid state. Therefore, the results in FIG. 1 were surprising. The stability results in FIG. 1 demonstrated that it was possible to stabilize a PTH(1-34) topical gel formulation at room temperature in the solid state.

### Example 11

### Evaluation of Room Temperature Topical Gel Formulations of PTH(1-34): Effect of Process Parameters and Excipients on Rehydration Time

Preliminary studies on rehydration time used water (see Table 13). Rehydration in 12% hexylene glycol, a humectant, decreased the rehydration time for a HEC 250HX lyophilized cake down to 20-30 minutes. Further experiments used 12% hexylene glycol for rehydration.

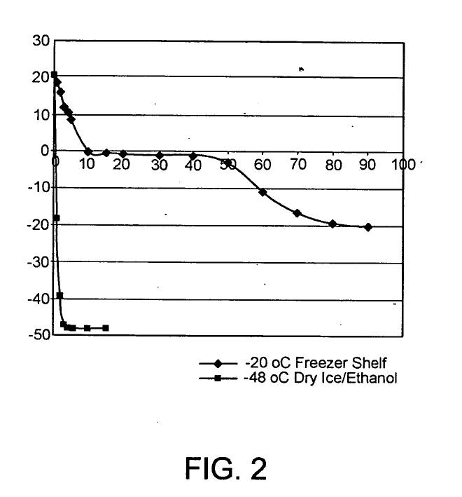

The freezing rate for lyophilized products can affect the cake structure. Freezing at −20° C. was the standard procedure used in this study. To investigate the effect of a faster freezing rate, samples were frozen in a dry ice/ethanol bath at −48° C. The freezing rates for a 1.125% HEC 250HX gel using both freezing conditions shown in FIG. 2.

The cooling rates for the two freezing methods were quite different. The lower temperature and higher thermal conductivity of the dry ice bath dramatically increased the freezing rate relative to that of the −20° C. shelf conditions.

Samples from both freezing methods were lyophilized. During the lyophilization process, all samples from the ice bath freezing collapsed into dense cakes. During rehydration, the ice bath frozen cakes were much slower to form gels. It took at least 1 hour to rehydrate the ice bath frozen cakes.

The effect of polymer concentration on rehydration time was also evaluated. To reduce lyophilization costs, the amount of water in a formulation can be decreased by freeze drying a more concentrated solution. The lower the water content, the faster lyophilization can be completed.

HEC 250HX gels at 1.125 and 2.25% polymer were prepared. The latter gel was more viscous than the former due to the higher polymer concentration. After freeze drying, cakes from both products were rehydrated so that the final polymer concentration was 1.125%. The lyophilized gels prepared with the more concentrated polymer solution required approximately 15-20 minutes longer to rehydrate than the more dilute formulation. The higher density of the concentrated polymer cake was likely a factor in the longer rehydration time.

Additives with a wide range of physical properties were screened to evaluate their effect on rehydration time for a lyophilized 1.125% HEC 250HX gel. The chemical classes included in this screen were polymers, surfactants, amino acids, colloidal powders, and salts. All additives were crystalline at room temperature. The use of amorphous excipients could lead to melting/cake collapse at elevated temperature. When evaluating chemical classes, priority was given to those included in the IIG for topicals. Excipients shown to be physically and/or chemically incompatible with PTH(1-34) were excluded (4). Salts containing polyvalent anions were also excluded to avoid the potential for precipitating PTH(1-34).

The results for additive screening are summarized in Table 18. Additives that had a positive effect (i.e., reduction in hydration time) have a check mark beside them. Those that are in the FDA's Inactive Ingredient Guide (IIG) for injectables but not topicals are italicized.

TABLE 18
Additives screened for improving hydration time for a lyophilized 1.125%
HEC 250 HX gel. Additives that had a positive effect (i.e., reduction
in hydration time) have a check mark beside them. Those that are in
the IIG for injectables but not topicals are italicized.

| | |
|---|---|
| Benzalkonium chloride | Potassium Chloride✓ |
| Colloidal SiO₂ | Sodium Chloride✓ |
| Glycine | Sodium Chloride + Mannitol |
| Lactose | Sodium Lactate✓ |
| Mannitol | Sorbitol |
| Methionine | Sorbitol + Mannitol |
| Polyvinyalcohol | Stearalkonium chloride |

While the excipients in Table 18 represent a broad range of physical properties, only salts like NaCl, KCl, and sodium lactate accelerated rehydration of the lyophilized gel. Using 0.1% of these salts reduced rehydration time down to a total of 15-25 minutes. Higher salt levels resulted in cake collapse.

Table 19 summarizes the compositions for two lyophilized PTH(1-34) gels prepared with approved salts for topical formulations. These freeze dried formulations were purged with nitrogen and stored at 40° C. for a pilot stability study.

TABLE 19
Composition of lyophilized gels containing salts that were used in a
study at 40° C.

| Batch Number | Wet Composition | Dry Composition | Comments |
|---|---|---|---|
| 80214-1 | 0.1% PTH(1-34) | ~7.5% PTH(1-34) | Stable cake |
| | 0.1% NaCl | | structure |
| | 1.125% HEC 250HX | | |
| 80214-2 | 0.1% PTH(1-34) | ~7.5% PTH(1-34) | Stable cake |
| | 0.1% Sodium lactate* | | structure |
| | 1.125% HEC 250HX | | |

*pH adjusted to 4.0 with HCl before freezing

After 1 month of accelerated storage, both formulations in Table 19 were >98.5% of their initial values, which was comparable to results in a pilot study with HEC and HEC/mannitol (see Table 16), suggesting that salts may be useful additives in the room temperature PTH(1-34) topical gel. In contrast, neat PTH(1-34) showed significant degradation after 1 month at 40° C. (see FIG. 1).

Endo et al. (U.S. Pat. No. 5,563,122) reported that salt improved the stability of PTH(1-34) injectable formulations in the solid state. However, the results in this topical gel study showed PTH(1-34) could be stabilized without salt (see FIG. 1). Using a salt in a lyophilized topical gel formulation was found useful for rehydration, a property not considered by Endo et al.

“Powdered” solid formulations were also evaluated. Reducing the lyophilized cake to a powder would increase the surface area and possibly reduce rehydration time. However, HEC cakes were somewhat difficult to mill due to their ductility. Lyophilized HEC cakes were reduced to a coarse powder by hand-cutting them using a razor blade and spatula. This powder was successfully added to a hexylene glycol solution to form a gel within 20-25 minutes.

An HEC powder with PTH(1-34) could also be prepared by spray drying. This process, while viable, is more challenging to develop since it requires optimization of the atomization, heating, and collection processes. Furthermore, if the powder is cohesive, it could form coarse agglomerates that are difficult to disperse.

Another option was explored to further decrease the rehydration time. A PTH(1-34) solution with mannitol was frozen then lyophilized to create a powder. This solution did not contain a polymer and the freeze dried mannitol formulation contained 9.1% PTH(1-34). A small amount of this powder, 0.11 g, was added to 9.89 g of a HEC gel base containing 1.125% HEC 250HX. The mannitol powder dissolved rapidly into the gel base with gentle stirring.

A sample of this lyophilized mannitol/PTH(1-34) formulation was purged with nitrogen and stored at 40° C. for 1 month. After 1 month of accelerated storage, this formulation was 98.9% of its initial value, which was comparable to results in a pilot study with HEC and HEC/mannitol (see Table 16). In contrast, neat PTH(1-34) showed significant degradation after 1 month at 40° C. (see FIG. 1).

Table 20 summarizes the three options evaluated in this study of room temperature stable topical gel formulations for PTH(1-34).

TABLE 20
Room temperature gel formulation options evaluated in this study.

| Option | Solid Component | Liquid Component |
|---|---|---|
| 1 | Lyophilized gel cake containing | Solution of humectant, buffer, |
| | PTH(1-34), polymer, plus | plus preservatives. |
| | additives. | |
| 2 | Lyophilized powder containing | Solution of humectant, buffer, |
| | PTH(1-34), polymer, plus | plus preservatives. |
| | additives. | |
| 3 | Lyophilized powder containing | Aqueous gel consisting of a |
| | PTH(1-34) plus small molecule | polymer, humectant, buffer, |
| | bulking/stabilizing agent (e.g., | plus preservatives. |
| | mannitol) | |

### Example 12

### Evaluation of Room Temperature Topical Gel Formulations: Compatibility Screening of Antioxidants

During preformulation studies, sodium thiosulfate and methionine were identified as effective antioxidants for PTH(1-34). When sodium thiosulfate was added to the gel formulation, a yellowish precipitate formed. Sodium thiosulfate was assayed in the gel formulations listed in Table 21 in the absence of PTH(1-34) in order to identify which excipients were incompatible with sodium thiosulfate. Gel formulations were prepared according to the method described in Example 8. LDPE dropper bottles (5 mL) were purchased from IntraPac Group and over wrap foil (FR-2175) was obtained from Covalence Coated Products. The appearance of all samples was noted initially and after one week of storage at 20° C. protected from light.

TABLE 21
Excipient compatibility with sodium thiosulfate. The sodium thiosulfate
level was fixed at 0.1% for all formulations. All formulations were
prepared as 100 gram batches.

| | | Appearance |
|---|---|---|
| Excipient(s) | Initial Appearance | after 7 days |
| 1% hydroxyethylcellulose | Clear and colorless | Clear and colorless |
| in water | | |
| 1% hydroxyethylcellulose | Clear and colorless | Yellow precipitate and |
| in pH 4 acetate buffer | | “sulfurous odor” |
| 12% hexylene glycol in | Clear and colorless | Clear and colorless |
| water | | |
| 12% hexylene glycol in pH | Clear and colorless | Yellow precipitate and |
| 4 acetate buffer | | “sulfurous odor” |
| 1% methylcellulose in | Clear and colorless | Clear and colorless |
| water | | |
| 1% methylcellulose in pH 4 | Clear and colorless | Yellow precipitate and |
| acetate buffer | | “sulfurous odor” |
| 1% hydroxypropylcellulose | Clear and colorless | Clear and colorless |
| in water | | |
| 1% hydroxypropyl | Clear and colorless | Clear and colorless |
| methylcellulose in water | | |
| 1% polyvinyl alcohol in | Clear and colorless | Clear and colorless |
| water | | |
| pH 4 acetate buffer | Clear and colorless | Yellow precipitate and |
| | | “sulfurous odor” |
| pH 4 citrate buffer | Clear and colorless | Yellow precipitate and |
| | | “sulfurous odor” |
| Water | Clear and colorless | Clear and colorless |
| 0.1 mM HCl | Clear and colorless | Yellow precipitate and |
| | | “sulfurous odor” |
| 0.1 mM HNO₃ | Clear and colorless | Yellow precipitate and |
| | | “sulfurous odor” |

The data in Table 21 suggested that the pH of the formulation was the primary contributor to precipitate formation. When sodium thiosulfate was combined with a wide range of polymers at neutral pH, no precipitation occurred.

### Example 13

### Evaluation of Room Temperature Topical Gel Formulations of PTH(1-34): Effect of Antioxidants on PTH(1-34) Stability

The stability of PTH(1-34) in room temperature topical gel formulations was assayed in two different antioxidant systems, one involving additions to the formulation itself and the other involving modifications to the packaging system. Formulations were prepared according to the method in Example 8 and packaged in LPDE vials according to Example 11 with the following additions. The three topical gel formulations for this study are listed in Table 22. The control formulation (Batch 70813-1) did not include any antioxidants. One formulation (Batch 70813-2) included 0.3% w/w methionine. The third formulation (Batch 708-1-N2) was identical to that of the control, but the LDPE vial was packaged in an oxygen-free environment in a nitrogen-purged foil over wrap pouch. The bottles for Batch 70813-1-N2 were purged with nitrogen before closing and immediately placed in a foil pouch that was purged with nitrogen before heat sealing it.

TABLE 22
Formulations prepared for stability studies (values in % w/w).

| | Batch | Batch | Batch |
|---|---|---|---|
| Component | 70813-1 | 70813-1-N2 | 70813-2 |
| PTH(1-34) | 0.10 | 0.10 | 0.10 |
| Hexylene glycol, NF | 12.00 | 12.00 | 12.00 |
| Hydroxyethylcellulose, NF (Natrosol ® | 1.25 | 1.25 | 1.25 |
| 250M PHARM) | | | |
| Phenoxyethanol, BP | 0.20 | 0.20 | 0.20 |
| Methylparaben, NF | 0.15 | 0.15 | 0.15 |
| Ethylparaben, NF | 0.05 | 0.05 | 0.05 |
| Methionine, USP | 0.00 | 0.00 | 0.30 |
| Acetic acid (glacial), USP | 0.041 | 0.041 | 0.041 |
| Sodium acetate (anhydrous), USP | 0.010 | 0.010 | 0.010 |
| Purified water, USP | q.s. | q.s. | q.s. |
| Nitrogen purge for bottle? | No | Yes | No |
| Nitrogen-purged foil over wrap pouch? | No | Yes | No |

Formulations were tested initially and after 1, 3, and 6 months of storage at 25° C. Refrigerated samples were only assayed at the 6 month time point since previous studies showed that degradation rates at these conditions were low.

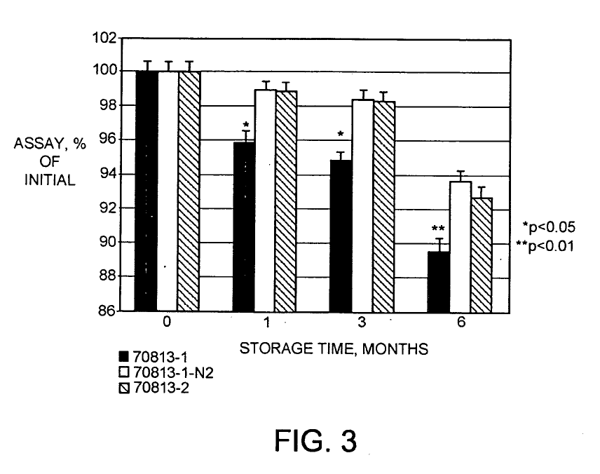

Assay values for PTH(1-34) formulations stored at 25° C. are summarized in FIG. 3. For all formulations, the assay value decreased with storage time. After one month of storage at 25° C., significantly less PTH remained in the control formulation (70813-1) (p<0.05) than the nitrogen-purged formulation (70813-1-N2) or the methionine formulation (70813-2). A similar result was seen at the third month of accelerated storage. At six months, the differences between the amount of PTH remaining in the control and the antioxidant groups had increased significantly (p<0.01). While the assay value for the nitrogen-purged formulation was slightly higher than the methionine formulation at the 6-month time point, the difference was not significant. In contrast, after six months of storage at 2-8° C., all three formulations retained greater than 98.5% of the initial PTH assay value.

With nitrogen, there are at least two options for packaging: foil over wrapped LDPE dropper bottles or foil laminate tubes with an ophthalmic tip.

Using a laminate tube may be a more practical option, but foil is required in the laminate to provide an oxygen barrier. The foil is laminated with one or more polymer layers on both sides in the final tube material. On the formulation contact side, the polymer layer isolates the formulation from the more reactive metal. There can be slight imperfections that allow minute quantities of metal to migrate into the formulation and for the formulation to have a very slight contact with the metal. Usually this is not a significant problem, but does require that the tube fabricator be selected based on compatibility data with the drug product.

### Example 14

### Evaluation of Percutaneous Absorption of Macromolecules in a Human Cadaver Skin Model: Materials and Methods

Radiolabeled Bovine Serum Albumin was acquired from American Radiolabeled Chemicals (St. Louis, Mo.) as ARI 0112 Albumin (bovine serum) [I¹²⁵] with a specific activity of 204 Ci/mmol (Lot number 080372). Radiolabeled Epidermal Growth Factor (murine) and Somatastatin were acquired from Perkin Elmer Life and Analytical Sciences (Boston, Mass.) as [¹²⁵I]-Epidermal Growth Factor (murine) and [¹²⁵I]Tyr11-Somatostatin 14 with specific activities of 2200 Ci/mmol (Lot number CC41180) and 2200 Ci/mmol (Lot number JX32880), respectively. Each stock isotope was either provided as, or reconstituted into 12.5 μL of distilled de-ionized water. This volume was added to 500 μL of the provided test formulations and mixed for uniformity. Replicate assessments were made from each final mixture to determine specific activity. The formulations evaluated are summarized in Table 23.

TABLE 23
Test formulations of macromolecules

| Formulation Identity | Lot Number | Specific Activity |
|---|---|---|
| BSA Gel 1.0 mg/mL | 80229-1 | 40,512 DPM/μL |
| Somatostatin Gel, 1.0 mg/mL | 80229-2 | 32,128 DPM/μL |
| EGF Gel, 0.1 mg/mL | 80229-3 | 40,078 DPM/μL |

All reagents used in this study were analytical reagent grade or better.

Skin Preparation. Percutaneous absorption was measured using the in vitro cadaver skin finite dose technique. Ex vivo human trunk skin without obvious signs of skin disease, obtained within 24-48 hours of death, was used in this study. Skin was dermatomed, prepared for cryopreservation, sealed in a water impermeable plastic bag, and stored at <−70° C. until the day of the experiment. Prior to use, it was thawed in ˜37° C. water, then rinsed in water to remove any adherent blood or other material from the surface.

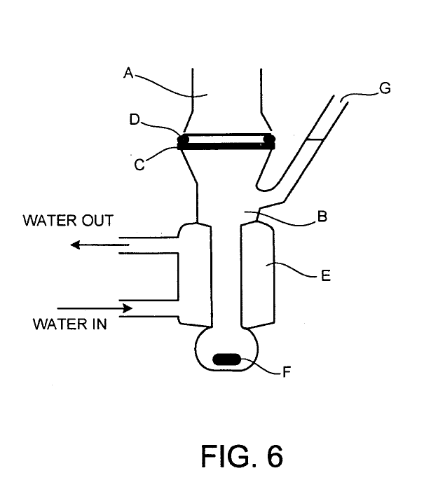

Skin from a single donor was cut into multiple smaller sections large enough to fit on static 1.0 cm² Franz diffusion cells (see FIG. 6). The dermal chamber was filled to capacity with a reservoir solution of phosphate-buffered isotonic saline (PBS), pH 7.4±0.1, and the epidermal cell (chimney) left open to ambient laboratory conditions. All cells were mounted in a diffusion apparatus in which the dermal bathing solution was stirred magnetically at approximately 600 RPM and the skin surface temperature maintained at 32.0° C.±1.0° C.

To assure the integrity of each skin section, its permeability to tritiated water was determined before application of the test products. Following a brief (0.5-1 hour) equilibrium period, ³H₂O (NEN, Boston, Mass., sp. Act. ˜0.5 μCi/mL) was layered across the top of the skin by dropper so that the entire exposed surface was covered (approximately 250-500 μL). After 5 minutes the ³H₂O aqueous layer was removed. At 30 minutes the receptor solution was collected and analyzed for radioactive content by liquid scintillation counting. Skin specimens in which absorption of ³H₂O was less than 1.56 μL-equ/cm² were considered acceptable.

Dosing and Sample Collection. Just prior to dosing, the reservoir solution was replaced with a fresh solution of 1× PBS and a pre-dose sample taken for analysis. The chimney was removed from the Franz Cell to allow full access to the epidermal surface of the skin. All formulations were then applied to the skin sections using a positive displacement pipette set to deliver 5 μL formulation/cm². The dose was spread across the surface with the Teflon® tip of the pipette. Five to ten minutes after application, the chimney portion of the Franz Cell was replaced. At pre-selected times after dosing (1, 2, 4, 8, 12, 24, 32, and 48 hours) the reservoir solution was removed in its entirety, replaced with fresh reservoir solution, and a predetermined volume aliquot saved for subsequent analysis.

In each study, spare cells were available which were not dosed but were used to monitor the appearance of substances diffusing out of the skin that might interfere with the analytic method.

After the last sample was collected, the surfaces were washed twice (0.5 mL volume each) with 1× PBS to collect un-absorbed formulation from the surface of the skin. Following the wash, the skin was tape stripped a sufficient number of times to collect the stratum corneum. The skin was then removed from the chamber and split into epidermis and dermis. All skin sections were dissolved overnight in Soluene-350® (Perkin Elmer).

Analytical Methods. Analysis of all formulations was by liquid scintillation counting. One-milliliter volumes of each receptor solution and each surface wash received 5-6 mL scintillation fluid or as needed. Each set of tape strips and each epidermal and dermal solution received 5-6 mL scintillation fluid or as needed. Samples were quantified for ¹²⁵I content by liquid scintillation spectroscopy using a PerkinElmer Tri-Carb 3100TR liquid scintillation counter. Each sample was counted for no less than 5 minutes each, in duplicate. Counts per minute (CPM) were automatically converted to decays per minute (DPM) using the external standard quench correction method.

Data were reported as microgram equivalents based on the ¹²⁵I content measured in the samples.

### Example 15

### Evaluation of Percutaneous Absorption of Macromolecules in a Human Cadaver Skin Model

The percutaneous absorption pharmacokinetics of Epidermal Growth Factor (EGF), Bovine Serum Albumin (BSA) and Somatostatin was assayed in ex vivo human skin, using an in vitro cadaver finite dose model. ¹²⁵I analogs of EGF, BSA and Somatostatin were prepared in a gel formulation according to the formulas in Tables 25, 26 and 27, respectively. The EGF and BSA formulations had a final pH of 6.8; the somatostatin formaulation had a final pH of 4.0.

Skin donor demographics are summarized in Table 24.

TABLE 24
Donor Demographics

| Donor ID | Age | Race | Sex | Integrity Test Result* |
|---|---|---|---|---|
| MB041107 | 47 | Caucasian | Female | 0.22 ± 0.04 |
| SD121306 | 54 | Caucasian | Male | 0.47 ± 0.09 |
| AS010207 | 55 | Caucasian | Male | 0.55 ± 0.21 |

*Results are reported as μL-equ ³H₂0; Acceptance ≦ 1.56 μl-equ/cm²

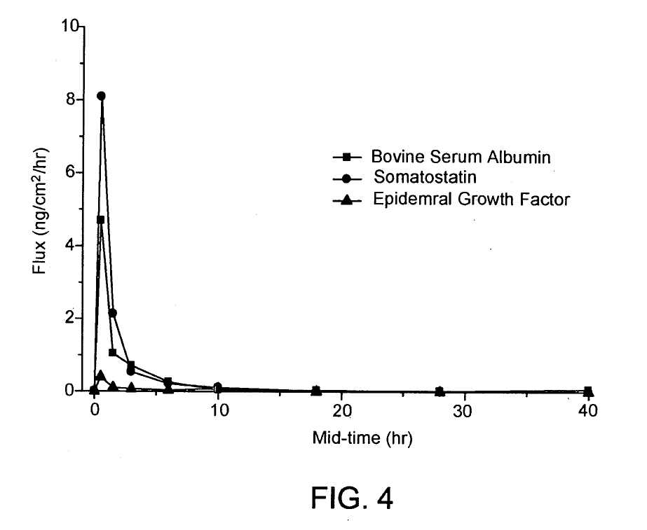

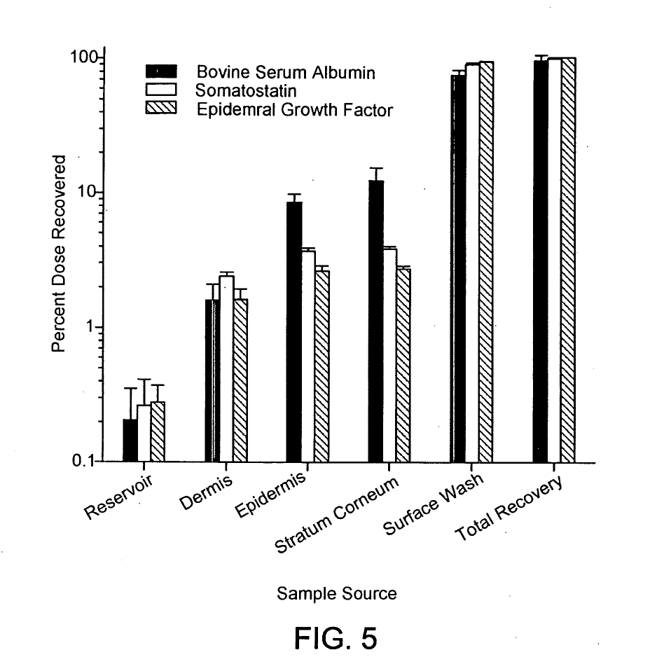

The results for the percutaneous absorption of [¹²⁵I]-EGF, BSA, and Somatostatin are summarized in Tables 24 and 25 and FIGS. 4 and 5.

TABLE 24
Mean Flux (ng/cm²/hr) Results: Across Donor Summary
Percutaneous Absorption of [¹²⁵I] BSA, Somatostatin
and EGF through Human Cadaver Skin over 48 hours from a Single
Application (Mean ± SE, n = 3 Donors).

| | Bovine Serum | | Epidermal Growth |
|---|---|---|---|
| Time | Albumin | Somatostatin | Factor |
| (hr)* | (Lot 80229-1) | (Lot 80229-2) | (Lot 80229-3) |
| 0.5 | 4.69 ± 3.93 | 8.09 ± 3.68 | 0.40 ± 0.21 |
| 1.5 | 1.04 ± 1.04 | 2.14 ± 1.89 | 0.11 ± 0.08 |
| 3.0 | 0.71 ± 0.43 | 0.54 ± 1.89 | 0.07 ± 0.06 |
| 6.0 | 0.26 ± 0.12 | 0.22 ± 0.20 | 0.04 ± 0.02 |
| 10.0 | 0.05 ± 0.05 | 0.11 ± 0.05 | 0.09 ± 0.06 |
| 18.0 | 0.04 ± 0.04 | 0.02 ± 0.02 | 0.01 ± 0.01 |
| 28.0 | 0.02 ± 0.02 | 0.00 ± 0.00* | 0.00 ± 0.00 |
| 40.0 | 0.06 ± 0.03 | 0.01 ± 0.00 | 0.01 ± 0.00 |

*Time as midpoint between samples.
** Zeros indicate results to be below the Lower Limit of Quantification (at or below background).

TABLE 25
Total Absorption and Mass Balance Results Across Skin Donors
Percutaneous Absorption and Penetration of [¹²⁵I] BSA,
Somatostatin and EGF into and through Intact Human Cadaver Skin
over 48 hours from a Single Application. Mean ± SE
as Percent of Applied Dose and Total Mass (μg).

| | Bovine Serum | | Epidermal |
|---|---|---|---|
| | Albumin | Somatostatin | Growth Factor |
| Parameter | (Lot 80229-1) | (Lot 80229-2) | (Lot 80229-3) |
| Total Absorption (μg) | 0.010 ± 0.007 | 0.013 ± 0.007 | 0.001 ± 0.000 |
| Dermis (μg) | 0.079 ± 0.024 | 0.118 ± 0.008 | 0.008 ± 0.002 |
| Epidermis (μg) | 0.423 ± 0.060 | 0.180 ± 0.010 | 0.013 ± 0.001 |
| Stratum Corneum (μg) | 0.616 ± 0.157 | 0.190 ± 0.009 | 0.014 ± 0.001 |
| Surface Wash (μg) | 3.788 ± 0.357 | 4.561 ± 0.118 | 0.479 ± 0.005 |
| Total Absorption (%) | 0.205 ± 0.148 | 0.234 ± 0.149 | 0.278 ± 0.095 |
| Dermis (%) | 1.607 ± 0.487 | 2.410 ± 0.169 | 1.629 ± 10.311 |
| Epidermis (%) | 8.610 ± 1.230 | 3.697 ± 0.200 | 2.646 ± 0.242 |
| Stratum Corneum (%) | 12.552 ± 3.202 | 3.886 ± 0.180 | 2.773 ± 0.131 |
| Surface Wash (%) | 77.182 ± 7.273 | 93.493 ± 2.412 | 98.145 ± 1.046 |
| Total Recovery (%) | 100.155 ± 10.321 | 103.749 ± 2.731 | 105.471 ± 0.688 |

The data in Tables indicated that radiolabeled iodine (¹²⁵I) from the EGF, BSA and Somatostatin penetrated into and, through human skin in vitro at about 1 to 13 nanograms/cm². Mass balance indicated that about 100%-105% of the applied dose was recovered across all formulations. The majority of the applied dose for each radiolabeled molecule was recovered in the surface wash, with decreasing levels recovered in the stratum corneum the epidermis and the dermis, respectively. Relatively higher percentages of BSA were recovered in the stratum corneum and epidermis than were recovered for Somatostatin and EGF in the same fractions.

Actual penetration into the reservoir solution must be viewed with caution as that which is penetrating may not necessarily be representing the fully intact molecule but rather free radiolabel or degradation fractions of the original molecule.

TABLE 26
EGF gel formulation

| Component | Function | % w/w |
|---|---|---|
| EGF | Active | 0.01 |
| Hexylene glycol, NF¹ | Solvent/Humectant | 12.00 |
| Hydroxyethylcellulose, NF | Gelling agent | 1.25 |
| Phenoxyethanol, BP | Preservative | 0.20 |
| Methylparaben, NF | Preservative | 0.15 |
| Ethylparaben, NF | Preservative | 0.05 |
| Acetic acid (glacial), USP | Buffer | 0.041 |
| Sodium acetate (anhydrous), USP | Buffer | 0.010 |
| Purified water, USP | Diluent | q.s. |

TABLE 27
BSA gel formulation

| Component | Function | % w/w |
|---|---|---|
| BSA | Active | 0.1 |
| Hexylene glycol, NF¹ | Solvent/Humectant | 12.00 |
| Hydroxyethylcellulose, NF | Gelling agent | 1.25 |
| Phenoxyethanol, BP | Preservative | 0.20 |
| Methylparaben, NF | Preservative | 0.15 |
| Ethylparaben, NF | Preservative | 0.05 |
| Acetic acid (glacial), USP | Buffer | 0.041 |
| Sodium acetate (anhydrous), USP | Buffer | 0.010 |
| Purified water, USP | Diluent | q.s. |

TABLE 28
Somatostatin gel formulation

| Component | Function | % w/w |
|---|---|---|
| Somatostatin | Active | 0.1 |
| Hexylene glycol, NF¹ | Solvent/Humectant | 12.00 |
| Hydroxyethylcellulose, NF | Gelling agent | 1.25 |
| Phenoxyethanol, BP | Preservative | 0.20 |
| Methylparaben, NF | Preservative | 0.15 |
| Ethylparaben, NF | Preservative | 0.05 |
| Acetic acid (glacial), USP | Buffer | 0.041 |
| Sodium acetate (anhydrous), USP | Buffer | 0.010 |
| Purified water, USP | Diluent | q.s. |

The present invention is not to be limited in scope by the specific embodiments described, which are intended as single illustrations of individual aspects of the invention. Functionally equivalent methods and components are within the scope of the invention. Indeed, various modifications of the invention, in addition to those shown and described herein, will become apparent to those skilled in the art from the foregoing description and accompanying drawings using no more than routine experimentation.

What is claimed is:

1. A topical composition comprising: (a) a therapeutically effective amount of a parathyroid hormone (PTH), a PTH-like hypercalcemic factor (CFF), or a biologically active variant of PTH or CFF, and (b) a vehicle comprising a polymer or a cationic liposome.

2. The topical composition of claim 1, further comprising a buffer.

3. The topical composition of claim 2, wherein the buffer comprises acetic acid, citric acid, lactic acid, or a combination thereof.

4. The topical composition of claim 3, wherein the composition is stable at room temperature.

5. The topical composition of any of claims 1-4, wherein the PTH comprises hPTH(1-84), hPTH(1-41), hPTH(1-38), hPTH(1-37), hPTH(1-34), hPTH(3-34), hPTH(5-34), hPTH(5-36), or hPTH(7-34).

6. The topical composition of claim 5, wherein the PTH comprises hPTH(1-34).

7. The topical composition of any of claims 1-6, wherein the polymer is a cellulose-based polymer.

8. The topical composition of any of claims 1-6, wherein the polymer comprises a hydroxyethylcellulose, a hydroxypropylcellulose, a methylcellulose, a carboxymethyl-cellulose, a hydroxypropyl methylcellulose, a polyvinyl alcohol, a polyvinylpyrrolidone, an alginate, a chitosan, or a carbomer.

9. The topical composition of claim 8, wherein the polymer comprises a hydroxyethylcellulose.

10. The topical composition of any of claims 1-9, wherein the polymer is a cationic or uncharged polymer.

11. The topical composition of any of claims 1-9, wherein the cationic liposome comprises a cholesterol or a derivative thereof.

12. The topical composition of any of claims 1-9, wherein the cationic liposome comprises a cationic surfactant.

13. The topical composition of any of claims 1-9, wherein the cationic liposome comprises, but is not limited to, a benzalkonium chloride, a stearalkonium chloride, a cetyl pyridinium chloride, a 2-dioleoyl-3-trimethyl ammonium propane, or a dimethyl dioctadecyl ammonium bromide.

14. The topical composition of any of claims 1-13, further comprising one or more of: an antioxidant; a chelating agent; an anti-microbial agent; a volatile solvent; a non-volatile solvent; an emollient; a humectant; and a stabilizer.

15. The topical composition of claim 14, wherein the antioxidant comprises sodium thiosulfate or methionine.

16. The topical composition of claim 14, wherein the chelating agent comprises EDTA.

17. The topical composition of claim 14, wherein the anti-microbial agent comprises phenoxyethanol, methylparaben, ethylparaben, or propylparaben.

18. The topical composition of claim 14, wherein the volatile solvent comprises water, ethanol, methylene chloride or isopropyl alcohol.

19. The topical composition of claim 14, wherein the non-volatile solvent comprises hexylene glycol, diethylene glycol monoethyl ether, glycerin, polyethylene glycol, dimethyl isosorbide, propylene carbonate, propylene glycol, or 1,2,6-trihydroxyhexane.

20. The topical composition of claim 14, wherein the humectant comprises hexylene glycol.

21. The topical composition of claim 14, wherein the stabilizer comprises sorbitol, mannitol, raffinose, trehalose, or lactose.

22. A topical composition comprising: (a) a therapeutically effective amount of a macromolecule having a molecular weight of about 1,500 daltons to about 1,000,000 daltons and (b) a polymer.

23. The topical composition of claim 22, further comprising one or more of: a solvent/humectant; a preservative; a buffer; and a diluent.

24. The topical composition of claim 22 or claim 23, further comprising one or more of: an antioxidant; a chelating agent; an anti-microbial agent; an emollient; and a stabilizer.

25. The topical composition of any of claims 22-24, wherein the macromolecule is present at about 0.005 to 0.5% (w/w).

26. The topical composition of any of claims 22-25, wherein the macromolecule is a parathyroid hormone (PTH), a PTH-like hypercalcemic factor (CFF), or a biologically active variant of PTH or CFF.

27. The topical composition of claim 26, wherein the PTH is PTH(1-34).

28. The topical composition of any of claims 22-25, wherein the macromolecule is an interferon, a growth factor, an interleukin, cyclosporine, a macrolide immunosuppressant, heparin, a hormone, or a glucosamine.

29. A kit for preparing the topical composition of any of claims 1-21, the kit comprising: (a) a solid component comprising a stabilized, solid PTH formulation, wherein the solid component is substantially free of any liquid, (b) a liquid component comprising an aqueous buffer and, optionally, an excipient to stabilize the formulation, and (c) instructions for combining the solid component and the liquid component.

30. The kit of claim 29, wherein the solid component further comprises a stabilizer in solid form and/or a disintegrant in solid form.

31. The kit of claim 30, wherein the stabilizer comprises sorbitol, mannitol, raffinose, trehalose, or lactose.

32. The kit of claim 31, wherein the stabilizer comprises mannitol.

33. The kit of claim 29, wherein the disintegrant comprises alginic acid, carboxy-methylcellulose salts, croscarmellose sodium, guar gum, microcrystalline cellulose, pregelatizined starch, sodium alginate, or prolacilin sodium.

34. The kit of claim 24, wherein the disintegrant comprises pregelatinized starch.

35. The kit of any of claims 29-34, wherein the liquid component comprises an acid or a humectant and the solid component comprises a salt.

36. A method of treating a subject who has a skin condition, the method comprising topically administering to the subject, on an area of the affected skin, the composition of any of claims 1-28 or a composition made by mixing the components of the kit of any of claims 29-35.

37. The method of claim 36, wherein the subject is a human.

38. The method of claim 36 or claim 37, wherein the skin condition is psoriasis.

39. The method of claim 37 or claim 38, wherein the skin condition is acne, comedones, polymorphonuclear leukocytes, rosacea, nodulocystic acne, acne conglobata, senile acne and secondary acne such as solar, medication-related, professional acne, ichthyosis, ichthyosiform states, Darier's disease, palmoplantar keratoderma, leukoplasias and leukoplasiform states, and cutaneous or mucous (buccal) lichen.

40. The method of any of claims 36-39 further comprising the step of identifying a subject in need of treatment.
